(19) United States
(12) Patent Application Publication
(10) Pub. No.: US 2025/0210192 A1
(43) Pub. Date: Jun. 26, 2025

(54) METHODS FOR DISEASE DETECTION

(71) Applicant: AEENA DX, INC., Mountain View, CA (US)

(72) Inventors: Shivani NAUTIYAL, Mountain View, CA (US); Nathan HUNT, Mountain View, CA (US); Jim VEITCH, Mountain View, CA (US)

(21) Appl. No.: 18/844,682

(22) PCT Filed: Mar. 7, 2023
(86) PCT No.: PCT/US2023/014738

§ 371 Date: Sep. 6, 2024

Related U.S. Application Data
(60) Provisional application No. 63/428,897, filed on Nov. 30, 2022.
(60) Provisional application No. 63/390,929, filed on Jul. 20, 2022.
(60) Provisional application No. 63/333,711, filed on Apr. 22, 2022.
(60) Provisional application No. 63/317,794, filed on Mar. 8, 2022.

(51) Int. Cl. G16H 50/20; C12Q 1/6806; C12Q 1/6886; G16B 40/00; G16H 10/40

(52) U.S. Cl. CPC G16H 50/20; C12Q 1/6806; C12Q 1/6886; G16B 40/00; G16H 10/40

(57) ABSTRACT

Described herein are methods for analyzing and detecting diseases or condition in a subject. Also, described herein are methods for preserving samples for detection of diseases or condition in a subject. Wherein detecting the disease or condition includes a) detecting the presence of at least one analyte or measuring the abundance of the at least one analyte in a sample from the subject; and b) generating a score for the likelihood of the subject having the disease or condition or the subject developing the disease or condition

### CROSS-REFERENCE

This application claims the benefit of U.S. Provisional Application Ser. No. 63/317,794 filed on Mar. 8, 2022; U.S. Provisional Application Ser. No. 63/333,711 filed on Apr. 22, 2022; U.S. Provisional Application Ser. No. 63/390,929 filed on Jul. 20, 2022; and U.S. Provisional Application Ser. No. 63/428,897 filed on Nov. 30, 2022, the entirety of which is hereby incorporated by reference herein.

### BACKGROUND

Cancer is one of the most prevalent diseases, affecting millions of people. For example, about 1 in 8 women in the United States develops invasive breast cancer over the course of her lifetime. Early detection and early treatment can increase survival rates of cancer patients. However, cancer detection can be cumbersome and is prone to false positive or false negative test results.

### SUMMARY

Accordingly, there remains a need for methods of detecting diseases, such as cancer, in a biological sample, where the biological sample is obtained, processed, and analyzed with efficiency and accuracy. Described herein, in some aspects, is a method for detecting a disease or condition in a subject, the method comprising: detecting the presence of at least one analyte or measuring the abundance of the at least one analyte in a sample from the subject; and generating a score for the likelihood of the subject having the disease or condition or the subject developing the disease or condition, wherein the sample is from a site of sample collection that is different from a site of the disease or condition, and wherein the presence of the at least one analyte or the abundance of the at least one analyte in the sample correlates with the presence of the at least one analyte, the abundance of the at least one analyte in the site of the disease or condition, or a consequence of the disease or condition. In some embodiments, prior to the detecting, the method comprises preserving the sample. In some embodiments, the preserving the sample comprises contacting the sample with a preservative comprising at least one of the following: ethylenediaminetetraacetic acid (EDTA); an RNase inhibitor; an anti-microbial; a denaturing agent; an agent that inhibits nuclease activity; a sequestration agent; a buffering agent; a salt; an osmolyte; or a combination thereof. In some embodiments, the denaturing agent comprises a nucleic acid denaturing agent or a protein denaturing agent. In some embodiments, prior to detecting, the method further comprises fractionating the sample. In some embodiments, the fractionating comprises separating the sample into two or more subsets of sample. In some embodiments, at least one of the two or more subsets of sample comprises a cell-containing fraction, wherein the cell-containing fraction comprises a cell originating from the subject or a cell not originating from the subject. In some embodiments, the cell originating from the subject is a human cell. In some embodiments, the cell not originating from the subject is a non-human cell. In some embodiments, the non-human cell comprises microbial cells. In some embodiments, the non-human cell comprises bacterial cells. In some embodiments, the non-human cell comprises fungal cells. In some embodiments, the non-human cell comprises archaeal cells. In some embodiments, at least one of the two or more subsets of sample comprises a cell-free fraction. In some embodiments, the fractionating comprises centrifuging the sample or filtrating the sample. In some embodiments, the sample comprises a biofluid. In some embodiments, the biofluid comprises blood, serum, plasma, saliva, urine, sweat, tears, breast milk, colostrum, semen, or cerebrospinal fluid. In some embodiments, the biofluid comprises saliva. In some embodiments, the at least one analyte comprises a cell-free analyte. In some embodiments, the at least one analyte comprises a nucleic acid. In some embodiments, the nucleic acid comprises a cell-free RNA. In some embodiments, the nucleic acid comprises mRNA, small RNA, miRNA, snoRNA, snRNA, rRNAs, tRNA, siRNA, hnRNA, long non-coding RNA, shRNA, fragments thereof, or a combination thereof. In some embodiments, the at least one analyte comprises a polypeptide. In some embodiments, the polypeptide is a protein. In some embodiments, the polypeptide is a metabolite. In some embodiments, the at least one analyte comprises a small molecule. In some embodiments, the at least one analyte comprises a metabolite. In some embodiments, the at least one analyte comprises a cell. In some embodiments, wherein the detecting comprises sequencing the at least one analyte, wherein the at least one analyte comprises at least one nucleic acid. In some embodiments, the detecting comprises hybridizing the at least one nucleic acid with a probe. In some embodiments, the disease or condition is cancer. In some embodiments, the cancer is breast cancer. In some embodiments, the disease or condition is a neurological disease. In some embodiments, the disease or condition is an autoimmune disease. In some embodiments, the disease or condition is a metabolic disease. In some embodiments, the disease or condition is an endocrine disease. In some embodiments, the disease or condition is a digestive tract disease. In some embodiments, the disease or condition is an injury. In some embodiments, the disease or condition is pregnancy. In some embodiments, the score determines the origin of the disease or condition. In some embodiments, the at least one analyte is DNA or cell-free salivary RNA of the subject, and both genetic and transcriptomic analyses are used to detect the presence of the disease or condition in the subject. In some embodiments, multiple samples from the subject are processed using different versions of the workflow described herein. In some embodiments, the method further comprises collecting the sample from the subject.

Described herein, in some aspects, is a method for detecting a disease or condition in a subject, the method comprising: with a computer system comprising a hardware processor and a memory on which instructions are encoded to cause the hardware processor to perform the operations of: detecting the presence of at least one analyte or measuring the abundance of the at least one analyte in a sample from a subject; and generating a score for the likelihood of the subject having the disease or condition or the subject developing the disease or condition, wherein the sample is from a site of sample collection that is different from a site of the disease or condition, and wherein the presence of the at least one analyte or the abundance of the at least one analyte in the sample correlates with the presence of the at least one analyte, the abundance of the at least one analyte in the site of the disease or condition, or a consequence of the disease or condition. In some embodiments, the method further comprises a step of generating a machine learning model iteratively trained to detect the disease or condition in the sample. In some embodiments, the method further comprises a step of generating a machine learning model iteratively trained to generate the score for the likelihood of the subject having the disease or condition. In some embodiments, the method further comprises a step of generating a machine learning model iteratively trained to generate the score for the likelihood of the subject developing the disease or condition. In some embodiments, the machine learning model comprises at least one of a XGBoost algorithm, a logistic regression model and a random forest algorithm.

Described herein, in some aspects, is an apparatus for detecting a disease or condition in a subject, the apparatus comprising: a computer system comprising a hardware processor and a memory on which instructions are encoded to cause the hardware processor to perform the operations of: detecting the presence of at least one analyte or measuring the abundance of the at least one analyte in a sample acquired from the subject; and generating a score for the likelihood of the subject having the disease or condition or the subject developing the disease or condition, wherein the sample is from a site of sample collection that is different from a site of the disease or condition, and wherein the presence of the at least one analyte or the abundance of the at least one analyte in the sample correlates with the presence of the at least one analyte, the abundance of the at least one analyte in the site of the disease or condition, or a consequence of the disease or condition. In some embodiments, the hardware processor generates a machine learning model iteratively trained to detect the disease or condition in the sample. In some embodiments, the hardware processor generates a machine learning model iteratively trained to generate the score for the likelihood of the subject having the disease or condition. In some embodiments, the hardware processor generates a machine learning model iteratively trained to generate the score for the likelihood of the subject developing the disease or condition. In some embodiments, the machine learning model comprises at least one of a XGBoost algorithm, logistic regression model and a random forest algorithm.

In some embodiments, the disclosure herein provides a method for detecting the presence of a medical condition or disease for a subject, the method comprising: (a) optionally collecting a bodily fluid sample from a subject; (b) optionally, preserving the sample at the time of collection by addition of a preservative; (c) optionally, fractionating the sample; (d) optionally, adding a preservative to the fractionated sample; (e) selecting one or more analytes in the sample including, but are not limited to, nucleic acid transcripts or genomic regions of interest; (f) qualitatively or quantitatively detecting the selected analytes with an assay(s), wherein the assay(s) may include techniques involving, but are not limited to, biomolecule purification, biomolecule enrichment, biomolecule sequencing, PCR, quantitative PCR, isothermal amplification, mass spectrometry, antibody-based detection, or CRISPR and CRISPR-Cas systems, thereby generating data; and (g) using a computer to analyze the data to detect the presence or absence of the medical condition or disease or analyzing the data to generate a likelihood score for the medical condition or disease.

Disclosed herein are methods for detecting a disease or condition in a subject, wherein the methods comprise: optionally collecting a sample from the subject; detecting the presence of at least one analyte or measuring the abundance of the at least one analyte in the sample; and generating a score for the likelihood of the subject having the disease or condition or the subject developing the disease or condition, wherein the sample is collected from a site of sample collection that is different from a site of the disease or condition, and wherein the presence of the at least one analyte or the abundance of the at least one analyte in the sample correlates with the presence of the at least one analyte, the abundance of the at least one analyte in the site of the disease or condition, or a consequence of the disease or condition. In some embodiments, the methods, prior to b), comprise preserving the sample. In some embodiments, the preserving the sample comprises contacting the sample with a preservative comprising at least one of the following: ethylenediaminetetraacetic acid (EDTA); an RNase inhibitor; an anti-microbial; a denaturing agent; an agent that inhibits nuclease activity; a sequestration agent; a buffering agent; a salt; an osmolyte; or a combination thereof. In some embodiments, the denaturing agent comprises a nucleic acid denaturing agent or a protein denaturing agent. In some embodiments, the methods, prior to b), further comprise fractionating the sample. In some embodiments, the fractionating comprises separating the sample into two or more subsets of sample. In some embodiments, at least one of the two or more subsets of sample comprises a cell-containing fraction, wherein the cell-containing fraction comprises a cell originating from the subject or a cell not originating from the subject. In some embodiments, the cell originating from the subject is human cell. In some embodiments, the cell not originating from the subject is non-human cells. In some embodiments, the non-human cell comprises microbial cells. In some embodiments, the non-human cell comprises bacterial cells. In some embodiments, the non-human cell comprises fungal cells. In some embodiments, the non-human cell comprises archaeal cells. In some embodiments, at least one of the two or more subsets of sample comprises a cell-free fraction. In some embodiments, the fractionating comprises centrifuging the sample or filtrating the sample. In some embodiments, the sample comprises a bodily fluid sample. In some embodiments, the bodily fluid sample comprises a saliva sample. In some embodiments, the at least one analyte comprises a nucleic acid. In some embodiments, the at least one analyte comprises a cell-free analyte. In some embodiments, the nucleic acid comprises a cell-free RNA. In some embodiments, the nucleic acid comprises mRNA, small RNA, miRNA, snoRNA, snRNA, rRNAs, tRNA, siRNA, hnRNA, long non-coding RNA, shRNA, fragments thereof, or a combination thereof. In some embodiments, the at least one analyte comprises a polypeptide. In some embodiments, the polypeptide is a protein. In some embodiments, the polypeptide is a metabolite. In some embodiments, the at least one analyte comprises a small molecule. In some embodiments, the at least one analyte comprises a metabolite. In some embodiments, the at least one analyte comprises a cell. In some embodiments, b) comprises sequencing the at least one analyte comprising at least one nucleic acid. In some embodiments, b) comprises hybridizing the at least one analyte, such as a nucleic acid, with a probe. In some embodiments, the disease or condition is cancer. In some embodiments, the cancer is breast cancer. In some embodiments, the disease or condition is a neurological disease. In some embodiments, the disease or condition is an autoimmune disease. In some embodiments, the disease or condition is a metabolic disease. In some embodiments, the disease or condition is an endocrine disease. In some embodiments, the disease or condition is a digestive tract disease. In some embodiments, the disease or condition is an injury. In some embodiments, the disease or condition is pregnancy. In some embodiments, the score determines the origin of the disease or condition. In some embodiments, the at least one analyte is DNA or cell-free salivary RNA of the subject, and both genetic and transcriptomic analyses are used to detect the presence of the disease or condition in the subject. In some embodiments, more than one sample is collected from the patient and the samples are processed using different versions of the workflow described herein.

Disclosed herein are also methods for stabilizing nucleic acid in a sample, wherein the methods comprise: a) filtering the sample, such that nucleic acid flows through the filter and remains in the filtrate; and b) adding one or more protein denaturants to the sample and/or filtrate to inhibit nuclease activity. In some embodiments, the nucleic acid in the sample is purified. In some embodiments, the sample is a biofluid comprising blood, serum, plasma, saliva, urine, sweat, tears, breast milk, colostrum, semen, or cerebrospinal fluid. In some embodiments, the sample is filtered prior to addition of the one or more protein denaturants. In some embodiments, the sample is collected in or transferred to a device with a filtration unit comprising at least one filter, wherein the device comprises a prefiltration mechanism to prevent clogging of smaller pore size filters; one or more filters; multiple filters arranged in order by pore size to allow successively smaller species to pass through; or at least two filters of the same type; a mechanism for applying mechanical force, centrifugal force, vacuum, capillary action, or radial or axial flow for filtering the sample through the at least one filter. In some embodiments, the filtration is achieved using depth filtration. In some embodiments, the methods further comprise using a filtrate collection vessel prefilled with the one or more denaturants to allow for rapid nuclease inactivation upon contact with filtrate. In some embodiments, the filtered sample is removed or decanted from the device into a second vessel. In some embodiments, the filtered sample is removed or decanted from the device into a second vessel containing a preservative. In some embodiments, a collection unit or a filtration unit is separated from the filtrate collection vessel post-filtration to allow addition of the preservative. In some embodiments, the preservative is stored in an enclosure within a cap and released upon securing the cap onto the detached filtrate collection vessel. In some embodiments, the nucleic acid is stabilized at a temperature range of between about −20° C. and about 50° C. In some embodiments, the nucleic acid is stabilized for at least 5 days. In some embodiments, the at least one filter comprises low nucleic acid-binding material. In some embodiments, the at least one filter has a size cutoff at least about 0.1 μm, at least about 1 μm, at least about 5 μm, at least about 10 μm, at least about 15 μm, at least about 20 μm, or at least about 25 μm. In some embodiments, the at least one filter has a size cutoff at most about 0.1 μm, at most about 1 μm, at most about 5 μm, at most about 10 μm, at most about 15 μm, at most about 20 μm, or at most about 25 μm. In some embodiments, the at least one filter has a size cutoff between about 0.1 μm and about 25 μm. In some embodiments, the at least one filter retains a plurality of white blood cells. In some embodiments, the at least one filter retains a plurality of red blood cells. In some embodiments, the at least one filter retains a plurality of cells derived from solid tissues. In some embodiments, the at least one filter retains a plurality of microbes. In some embodiments, the at least one filter comprises synthetic material to minimize the introduction of contaminating nucleic acid. In some embodiments, the at least one or filter comprises biological material. In some embodiments, the filtering produces a cell-free biofluid or a cell-depleted biofluid. In some embodiments, the filtering produces cell-free plasma or cell-depleted plasma. In some embodiments, the filtering produces cell-free saliva or cell-depleted saliva. In some embodiments, the filtering produces cell-free urine or cell-depleted urine. In some embodiments, the nucleic acid is RNA. In some embodiments, the nucleic acid is DNA. In some embodiments, the one or more denaturants contain one or more chaotropic agents comprising detergent, urea, thiourea, guanidine thiocyanate, dodecylguanidine, dodine, or guanidine hydrochloride. In some embodiments, the one or more denaturants comprise the guanidine thiocyanate at a concentration of between about 30% and about 70%. In some embodiments, the retentate can be recovered and, biomolecules and cells contained within the retentate are preserved and analyzed.

Disclosed herein is an apparatus for detecting a disease or condition in a subject, the apparatus comprising: a computer system comprising a hardware processor and a memory on which instructions are encoded to cause the hardware processor to perform the operations of: detecting the presence of at least one analyte or measuring the abundance of the at least one analyte in a sample acquired from the subject; and generating a score for the likelihood of the subject having the disease or condition or the subject developing the disease or condition, wherein the sample is collected from a site of sample collection that is different from a site of the disease or condition, and wherein the presence of the at least one analyte or the abundance of the at least one analyte in the sample correlates with the presence of the at least one analyte, the abundance of the at least one analyte in the site of the disease or condition, or a consequence of the disease or condition.

Disclosed herein is a method for detecting a disease or condition in a subject, the method comprising: acquiring a sample from the subject; with a computer system comprising a hardware processor and a memory on which instructions are encoded to cause the hardware processor to perform the operations of: detecting the presence of at least one analyte or measuring the abundance of the at least one analyte in the sample; and generating a score for the likelihood of the subject having the disease or condition or the subject developing the disease or condition, wherein the sample is collected from a site of sample collection that is different from a site of the disease or condition, and wherein the presence of the at least one analyte or the abundance of the at least one analyte in the sample correlates with the presence of the at least one analyte, the abundance of the at least one analyte in the site of the disease or condition, or a consequence of the disease or condition.

Disclosed herein is a method for detecting a disease or condition in a subject, the method comprising: a) detecting the presence of at least one analyte or measuring the abundance of the at least one analyte in a sample from the subject; and b) generating a score for the likelihood of the subject having the disease or condition or the subject developing the disease or condition, wherein the sample is from a site of sample collection that is different from a site of the disease or condition, and wherein the presence of the at least one analyte or the abundance of the at least one analyte in the sample correlates with the presence of the at least one analyte, the abundance of the at least one analyte in the site of the disease or condition, or a consequence of the disease or condition. In some embodiments, prior to a), the method comprises preserving the sample. In some embodiments, the preserving the sample comprises contacting the sample with a preservative comprising at least one of the following: ethylenediaminetetraacetic acid (EDTA); an RNase inhibitor; an anti-microbial; a denaturing agent; an agent that inhibits nuclease activity; a sequestration agent; a buffering agent; a salt; an osmolyte; or a combination thereof. In some embodiments, the denaturing agent comprises a nucleic acid denaturing agent or a protein denaturing agent. In some embodiments, prior to a), the method further comprises fractionating the sample. In some embodiments, the fractionating comprises separating the sample into two or more subsets of sample. In some embodiments, at least one of the two or more subsets of sample comprises a cell-containing fraction, wherein the cell-containing fraction comprises a cell originating from the subject or a cell not originating from the subject. In some embodiments, the cell originating from the subject is a human cell. In some embodiments, the cell not originating from the subject is a non-human cell. In some embodiments, the non-human cell comprises microbial cells. In some embodiments, the non-human cell comprises bacterial cells. In some embodiments, the non-human cell comprises fungal cells. In some embodiments, the non-human cell comprises archaeal cells. In some embodiments, at least one of the two or more subsets of sample comprises a cell-free fraction. In some embodiments, the fractionating comprises centrifuging the sample or filtrating the sample. In some embodiments, the sample comprises a biofluid. In some embodiments, the biofluid comprises blood, serum, plasma, saliva, urine, sweat, tears, breast milk, colostrum, semen, or cerebrospinal fluid. In some embodiments, the biofluid comprises saliva. In some embodiments, the at least one analyte comprises a cell-free analyte. In some embodiments, the at least one analyte comprises a nucleic acid. In some embodiments, the nucleic acid comprises a cell-free RNA. In some embodiments, the nucleic acid comprises mRNA, small RNA, miRNA, snoRNA, snRNA, rRNAs, tRNA, siRNA, hnRNA, long non-coding RNA, shRNA, fragments thereof, or a combination thereof. In some embodiments, the at least one analyte comprises a polypeptide. In some embodiments, the polypeptide is a protein. In some embodiments, the polypeptide is a metabolite. In some embodiments, the at least one analyte comprises a small molecule. In some embodiments, the at least one analyte comprises a metabolite. In some embodiments, the at least one analyte comprises a cell. In some embodiments, a) comprises sequencing the at least one analyte, wherein the at least one analyte comprises at least one nucleic acid. In some embodiments, a) comprises hybridizing the at least one nucleic acid with a probe. In some embodiments, the disease or condition is cancer. In some embodiments, the cancer is breast cancer. In some embodiments, the disease or condition is a neurological disease. In some embodiments, the disease or condition is an autoimmune disease. In some embodiments, the disease or condition is a metabolic disease. In some embodiments, the disease or condition is an endocrine disease. In some embodiments, the disease or condition is a digestive tract disease. In some embodiments, the disease or condition is an injury. In some embodiments, the disease or condition is pregnancy. In some embodiments, the score determines the origin of the disease or condition. In some embodiments, the at least one analyte is DNA or cell-free salivary RNA of the subject, and both genetic and transcriptomic analyses are used to detect the presence of the disease or condition in the subject. In some embodiments, multiple samples from the subject are processed using different versions of the workflow described herein. In some embodiments, the method further comprises collecting the sample from the subject.

Disclosed herein is a method for detecting a disease or condition in a subject, the method comprising: with a computer system comprising a hardware processor and a memory on which instructions are encoded to cause the hardware processor to perform the operations of: detecting the presence of at least one analyte or measuring the abundance of the at least one analyte in a sample from a subject; and generating a score for the likelihood of the subject having the disease or condition or the subject developing the disease or condition, wherein the sample is from a site of sample collection that is different from a site of the disease or condition, and wherein the presence of the at least one analyte or the abundance of the at least one analyte in the sample correlates with the presence of the at least one analyte, the abundance of the at least one analyte in the site of the disease or condition, or a consequence of the disease or condition. In some embodiments, the method further comprises a step of generating a machine learning model iteratively trained to detect the disease or condition in the sample. In some embodiments, the method further comprises a step of generating a machine learning model iteratively trained to generate the score for the likelihood of the subject having the disease or condition. In some embodiments, the method further comprises a step of generating a machine learning model iteratively trained to generate the score for the likelihood of the subject developing the disease or condition. In some embodiments, the machine learning model comprises at least one of a XGBoost algorithm, a logistic regression model and a random forest algorithm.

Also described herein is an apparatus for detecting a disease or condition in a subject, the apparatus comprising: a computer system comprising a hardware processor and a memory on which instructions are encoded to cause the hardware processor to perform the operations of: detecting the presence of at least one analyte or measuring the abundance of the at least one analyte in a sample acquired from the subject; and generating a score for the likelihood of the subject having the disease or condition or the subject developing the disease or condition, wherein the sample is from a site of sample collection that is different from a site of the disease or condition, and wherein the presence of the at least one analyte or the abundance of the at least one analyte in the sample correlates with the presence of the at least one analyte, the abundance of the at least one analyte in the site of the disease or condition, or a consequence of the disease or condition. In some embodiments, the hardware processor generates a machine learning model iteratively trained to detect the disease or condition in the sample. In some embodiments, the hardware processor generates a machine learning model iteratively trained to generate the score for the likelihood of the subject having the disease or condition. In some embodiments, the hardware processor generates a machine learning model iteratively trained to generate the score for the likelihood of the subject developing the disease or condition. In some embodiments, the machine learning model comprises at least one of a XGBoost algorithm, logistic regression model and a random forest algorithm.

Also described herein is a device for collecting and stabilizing an analyte in a sample, the device comprising: a) a sample collection vessel for collecting the sample; b) a filtration unit in fluid communication with the sample collection vessel, the filtration unit comprising at least one filter for filtering the sample to produce a filtrate; c) a filtrate collection vessel in fluid communication with the filtration unit for collecting the filtrate and contacting the filtrate with a preservative. In some embodiments, the filtration unit comprises multiple filters arranged in order by pore size to allow successively smaller species to pass through. In some embodiments, the filtration unit comprises at least two filters of the same type. In some embodiments, the device comprises a prefiltration mechanism to prevent clogging of smaller pore size filters. In some embodiments, the filtration unit comprises a single filter. In some embodiments, the at least one filter comprises a depth filter. In some embodiments, the at least one filter comprises an asymmetric filter. In some embodiments, the at least one filter comprises a microporous filter. In some embodiments, the at least one filter comprises low nucleic acid-binding material. In some embodiments, the at least one filter has a size cutoff of at least about 0.1 μm, at least about 1 μm, at least about 2 μm, at least about 3 μm, at least about 4 μm, at least about 5 μm, at least about 10 μm, at least about 15 μm, at least about 20 μm, at least about 25 μm, at least about 30, at least about 35 μm, at least about 40 μm, at least about 45 μm, at least about 50 μm, at least about 55 μm, at least about 60 μm, at least about 65 μm, at least about 70 μm, at least about 75 μm, at least about 80 μm, at least about 85 μm, at least about 90 μm, at least about 95 μm, or at least about 100 μm. In some embodiments, the at least one filter has a size cutoff at most about 0.1 μm, at most about 1 μm, at most about 2 μm, at most about 3 μm, at most about 4 μm, at most about 5 μm, at most about 10 μm, at most about 15 μm, at most about 20 μm, at most about 25 μm, at most about 30, at most about 35 μm, at most about 40 μm, at most about 45 μm, at most about 50 μm, at most about 55 μm, at most about 60 μm, at most about 65 μm, at most about 70 μm, at most about 75 μm, at most about 80 μm, at most about 85 μm, at most about 90 μm, at most about 95 μm, or at most about 100 μm. In some embodiments, the at least one filter has a size cutoff between about 0.1 μm and about 100 μm. In some embodiments, filter has a thickness of from about 50 μm to about 1000 μm, or from about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, or about 950 μm to about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 100 μm, such as from about 355 μm to about 560 μm, such as about 330 μm, such as from about 120 μm to about 170 μm, such as from about 230 μm to about 270 μm, such as from about 480 μm to about 640 μm. In some embodiments, the filter has a diameter of from about 10 mm to about 50 mm, such as from about 10 mm, about 15 mm, about 20 mm, about 25 mm, about 30 mm, about 35 mm, about 40 mm, or about 45 mm to about 15 mm, about 20 mm, about 25 mm, about 30 mm, about 35 mm, about 40 mm, about 45 mm, or about 50 mm. In some embodiments, the filtration unit comprises a filter stack height of from about 120 μm to about 10000 μm, such as from about 120 μm, about 150 μm, about 175 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1250 μm, about 1500 μm, about 1750 μm, about 2000 μm, about 2500 μm, about 3000 μm, about 3500 μm, about 4000 μm, about 4500 μm, about 5000 μm, about 5500 μm, about 6000 μm, about 6500 μm, about 7000 μm, about 7500 μm, about 8000 μm, about 8500 μm, about 9000 μm, or about 9500 μm to about 150 μm, about 175 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1250 μm, about 1500 μm, about 1750 μm, about 2000 μm, about 2500 μm, about 3000 μm, about 3500 μm, about 4000 μm, about 4500 μm, about 5000 μm, about 5500 μm, about 6000 μm, about 6500 μm, about 7000 μm, about 7500 μm, about 8000 μm, about 8500 μm, about 9000 μm, about 9500 μm, or about 10000 μm. In some embodiments, the at least one filter is hydrophilic or hydrophobic. In some embodiments, the at least one filter comprises polysulfone and/or polypropylene. In some embodiments, the at least one filter retains a plurality of white blood cells. In some embodiments, the at least one filter retains a plurality of red blood cells. In some embodiments, the at least one filter retains a plurality of cells derived from solid tissues. In some embodiments, the at least one filter retains a plurality of microbes. In some embodiments, the at least one filter comprises synthetic material to minimize the introduction of contaminating nucleic acid. In some embodiments, the at least one filter comprises biological material. In some embodiments, the at least one filter is free of biological material. In some embodiments, the biological material comprises cellulose. In some embodiments, the sample is a biofluid comprising blood, serum, plasma, saliva, urine, sweat, tears, breast milk, colostrum, semen, or cerebrospinal fluid. In some embodiments, the filtrate is a cell-free biofluid or a cell-depleted biofluid. In some embodiments, the filtrate is cell-free plasma or cell-depleted plasma. In some embodiments, the filtrate is cell-free saliva or cell-depleted saliva. In some embodiments, the filtrate is cell-free urine or cell-depleted urine. In some embodiments, the device further comprises a mechanism for applying mechanical force, centrifugal force, vacuum, capillary action, or radial or axial flow for filtering the sample through at least one of the at least one filter. In some embodiments, the mechanism is for applying mechanical force, centrifugal force, vacuum, capillary action, or radial or axial flow for filtering the sample through all filters in the device. In some embodiments, the mechanism comprises a plunger that engages with the sample collection vessel to push the sample through the filtration unit and into the filtrate collection vessel. In some embodiments, the plunger is integral with or separate from the sample collection vessel. In some embodiments, the sample collection vessel comprises a funnel, wherein the funnel is integral with or couplable to the sample collection vessel. In some embodiments, the filtrate collection vessel comprises the preservative. In some embodiments, the preservative comprises at least one of the following: ethylenediaminetetraacetic acid (EDTA); an RNase inhibitor; an anti-microbial; a denaturing agent; an agent that inhibits nuclease activity, a sequestration agent; a buffering agent; a salt; an osmolyte; or a combination thereof. In some embodiments, the denaturing agent comprises a nucleic acid denaturing agent or a protein denaturing agent. In some embodiments, the agent that inhibits nuclease activity comprises one or more protein denaturants, EDTA, detergents such as SDS, aurintricarboxylic acid (ATA), chelating agents, or combinations thereof. In some embodiments, the one or more protein denaturants comprise one or more chaotropic agents comprising detergent, urea, thiourea, guanidine thiocyanate, dodecylguanidine, dodine, or guanidine hydrochloride. In some embodiments, the one or more protein denaturants comprise the guanidine thiocyanate at a concentration of between about 30% and about 70%. In some embodiments, the filtrate collection vessel is detachable from the filtration unit. In some embodiments, the device further comprises a cap for the detached filtrate collection vessel. In some embodiments, the cap comprises an enclosure storing a preservative that is released upon securing the cap onto the detached filtrate collection vessel. In some embodiments, the device further comprises a second vessel for decanting the filtrate. In some embodiments, the second vessel comprises a preservative. In some embodiments, the device stabilizes the analyte at a temperature range of between about −20° C. and about 50° C. In some embodiments, the analyte is stabilized for at least 5 days. In some embodiments, the at least one analyte comprises a cell-free analyte. In some embodiments, the at least one analyte comprises a nucleic acid. In some embodiments, the nucleic acid comprises a cell-free RNA. In some embodiments, the nucleic acid comprises mRNA, small RNA, miRNA, snoRNA, snRNA, rRNAs, tRNA, siRNA, hnRNA, long non-coding RNA, shRNA, fragments thereof, or a combination thereof. In some embodiments, the at least one analyte comprises a polypeptide. In some embodiments, the polypeptide is a protein. In some embodiments, the polypeptide is a metabolite. In some embodiments, the at least one analyte comprises a small molecule. In some embodiments, the at least one analyte comprises a metabolite. In some embodiments, the at least one analyte comprises a cell. In some embodiments, the filtration unit is detachable for retentate recovery so that biomolecules and cells contained within the retentate can be preserved and analyzed. In some embodiments, the at least one filter reduces the viscosity of the filtrate and compared to the sample. In some embodiments, the at least one filter removes mucins from the sample.

Also described herein is a kit comprising the device described herein and at least one of: a) a plunger; b) a cap for the filtrate collection vessel; c) a funnel; and/or d) a preservative.

Also described herein is a method for stabilizing an analyte in a sample, the method comprising applying the sample to the sample collection vessel of the device or the kit described herein, filtering the sample through the filtration unit, and contacting the filtrate with the preservative.

Also described herein is a analyte stabilized by the method described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows a flow diagram providing an overview of workflow steps.

FIG. 2 shows a flow diagram providing an overview of workflow steps.

FIG. 3 shows a flow chart for identifying tissue specific transcripts and tissue enriched transcripts present in a patient sample.

FIG. 4 shows a diagram depicting that tissue specific transcripts in saliva demonstrate a potential for broad disease detection. Specifically, the diagram depicts tissue and sample hierarchical clustered heat map in which red features denote higher levels of mRNA overlap between tissue-specific transcripts and cell-free saliva from an individual, while blue features denote lower levels of mRNA overlap.

FIG. 5 shows a diagram depicting that tissue enriched transcripts in saliva demonstrate a potential for broad disease detection. Specifically, the diagram depicts tissue and sample hierarchical clustered heat map in which red features denote higher levels of mRNA overlap between tissue-enriched transcripts and cell-free saliva from an individual, while blue features denote lower levels of mRNA overlap.

FIG. 6 shows a diagram illustrating the contents of whole saliva.

FIG. 7 shows a diagram depicting a device concept with a procedure.

FIG. 8 shows a diagram illustrating saliva collection and preservation preliminary experiments. In the experiments, saliva filtration for removal of human cell was followed by preservation of the resulting cell-free saliva by addition of a chaotropic preservative. BioAnalyzer analysis was used for quantitation and to assess degradation. ACTB, FOSL2, and NAMPT transcript levels were monitored by RT-qPCR to assess degradation.

FIG. 9 shows preliminary data for the nucleic acid preservation aspects described herein. Sample filtering provided similar yields to clearing by centrifugation. The addition of preservative protected the transcripts for four days.

FIG. 10 shows a graph illustrating that human sequence coverage improves with exome capture. A pilot experiment was conducted and demonstrated that exome enrichment using hybrid capture resulted in significantly greater coverage of GENCODE genes. Future optimization of hybridization conditions and probes may further improve performance.

FIG. 11 shows a diagram illustrating the bioinformatics pipeline. Human alignments were performed using hg38. Microbial alignments were performed using the Human Oral Microbiome Database.

FIG. 12 shows a diagram illustrating the landscape of sequencing reads. Reads spanned a large range across samples. HOM reads were consistent across samples and made up 20% of the total reads. Greatest variability was seen for the hg38 mapped reads: about 100-fold difference between highest and lowest coverage.

FIG. 13 shows a diagram illustrating the final reads post alignment. Deduplication reduced the number of reads by about 8-fold but removed amplification bias. “Assigned to gene” represents final features.

FIG. 14A and FIG. 14B show graphs illustrating the final feature counts across 332 samples.

FIG. 15A and FIG. 15B show two diagrams illustrating sequence QC of final features. GC profiles were consistent across samples. Coverage through the length of the gene was consistent across samples. Bias was observed towards higher coverage on 5′ end of transcript.

FIG. 16 shows a chart depicting the 301 patient NGS study, including 115 breast cancer patients and 186 non-cancer patients.

FIG. 17A and FIG. 17B show two graphs depicting the classification results. In FIG. 17A, a machine learning classifier using the 20 cancer and 20 non-cancer cohorts showed good performance, with an AUC of 0.763. A 10-fold cross validation (CV) was performed. In FIG. 17B, classification was performed using randomized disease labels. The mean AUC across 100 iterations was 0.486, suggesting that the result with correct labels was non-random. Increasing the hg38 mapped reads through assay optimization may generalize this performance across all samples.

FIG. 18 shows a table illustrating that Gene Set Enrichment Analysis showed multiple gene sets enriched in the cancer group.

FIG. 19 shows a diagram depicting the ensemble classifier, including logistic regression, random forest, and XGBoost.

FIG. 20 shows a diagram illustrating a 10-fold cross validation which was performed 100 times with shuffling.

FIG. 21A illustrates performance of logistic regression model using 20 breast cancer and 20 noncancer patients. FIG. 21B illustrates the highest coefficient 157 genes that contribute to the performance of the classifier. A positive coefficient was indicative of the gene generally being upregulated in cancer patients, and a negative coefficient was indicative of down regulation. FIG. 21C illustrates classifier discrimination as a function of feature exclusion. Discrimination was lost after removal of about 250 top features.

FIG. 22 shows a non-limiting example of a computing device; in this case, a device with one or more processors, memory, storage, and a network interface.

FIG. 23 shows a non-limiting example of a web/mobile application provision system; in this case, a system providing browser-based and/or native mobile user interfaces.

FIG. 24 shows a non-limiting example of a cloud-based web/mobile application provision system; in this case, a system comprising an elastically load balanced, auto-scaling web server and application server resources as well synchronously replicated databases.

FIG. 25A-E show improvement of preserving nucleic acid in saliva sample. FIG. 25A shows degradation of RNA encoding Actin B over three days. FIG. 25B shows a schematic diagram outlining a seven-day nucleic acid stability study of a method described herein for preserving nucleic acid. FIG. 25C shows an exemplary profile from one donor over seven days, illustrating that the filter and preservative combinatory condition showed the most stable profile over seven days. FIG. 25D shows the preservation of spike-in control being preserved over seven days. FIG. 25E shows stability of endogenous transcripts over seven days.

### DETAILED DESCRIPTION

### Overview

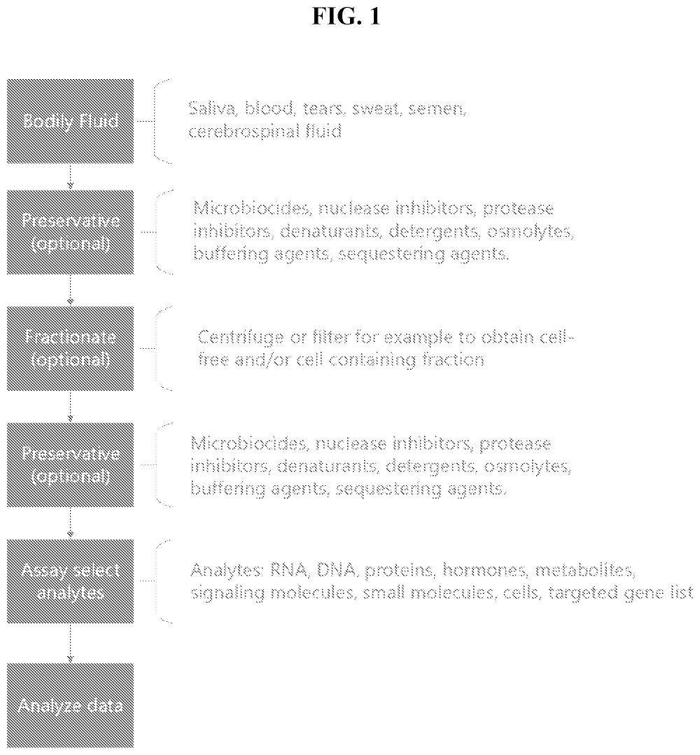

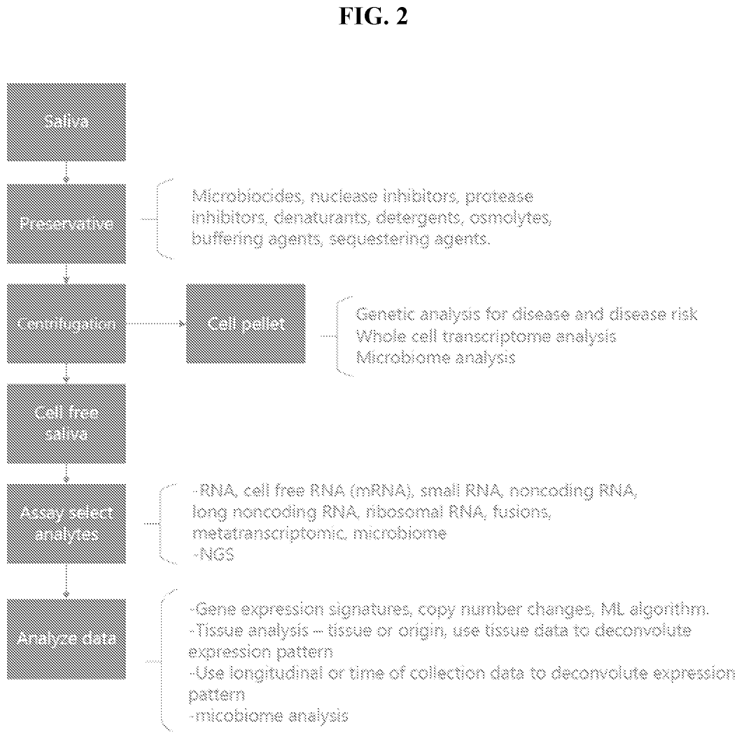

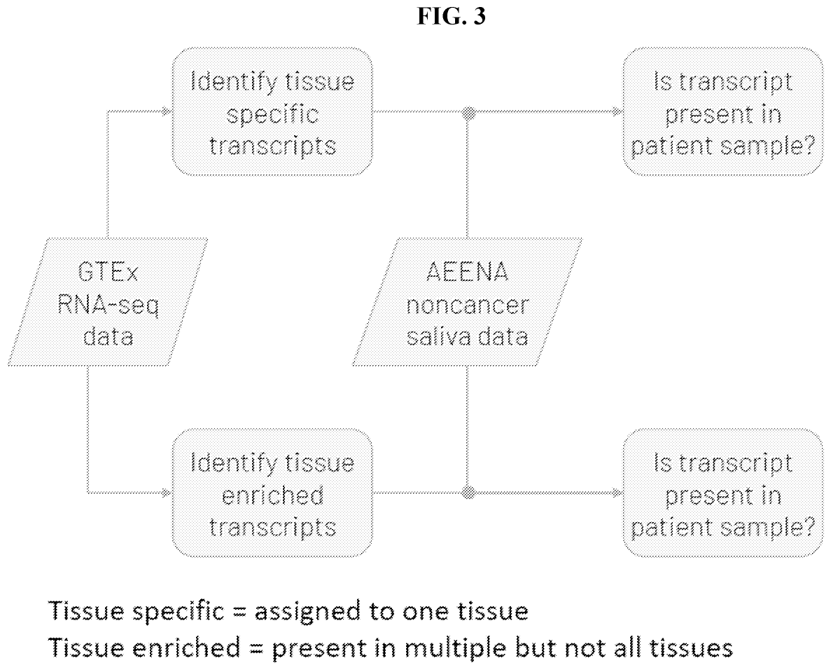

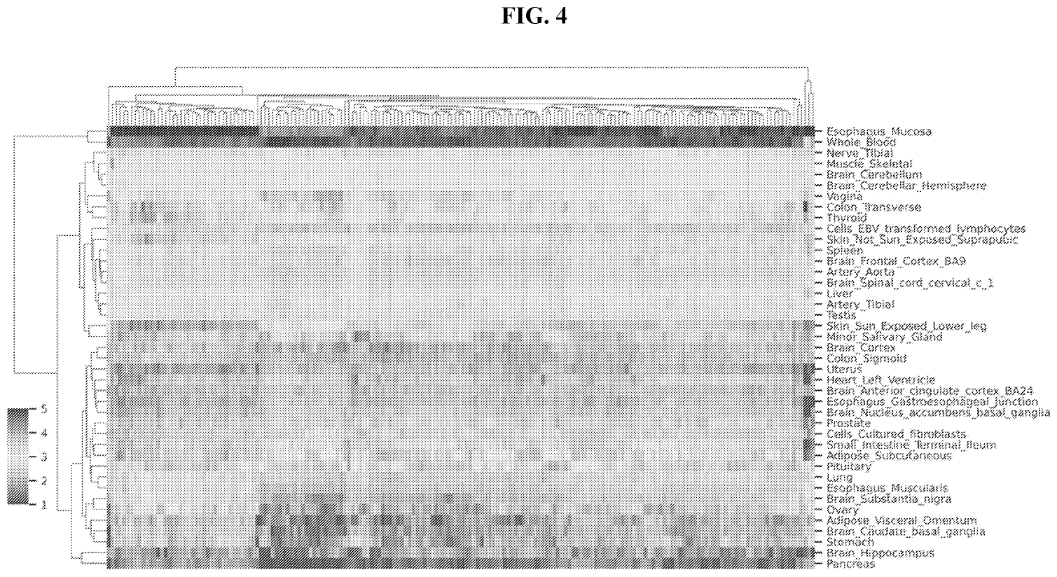

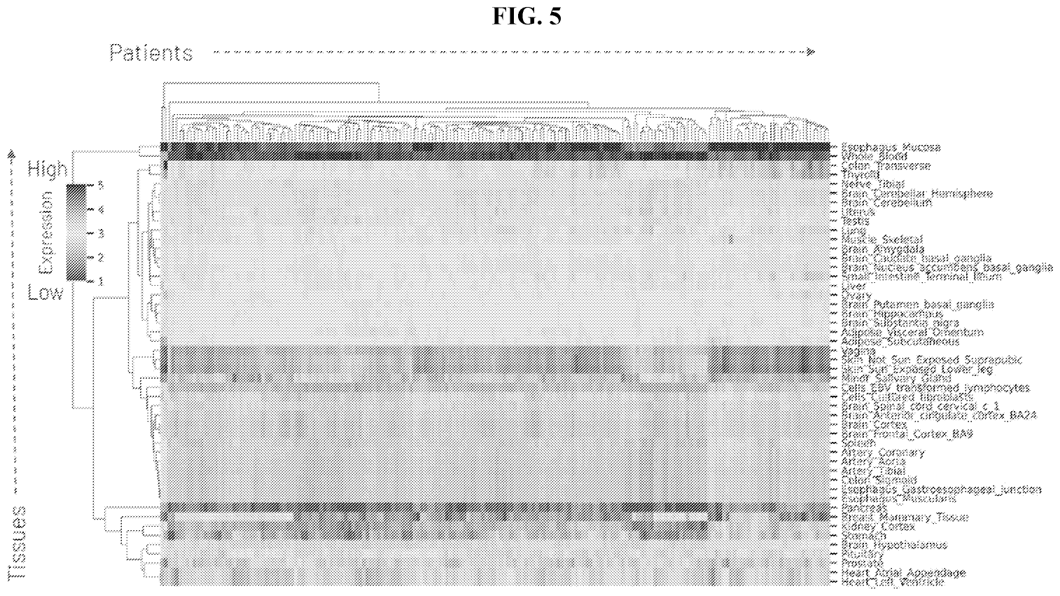

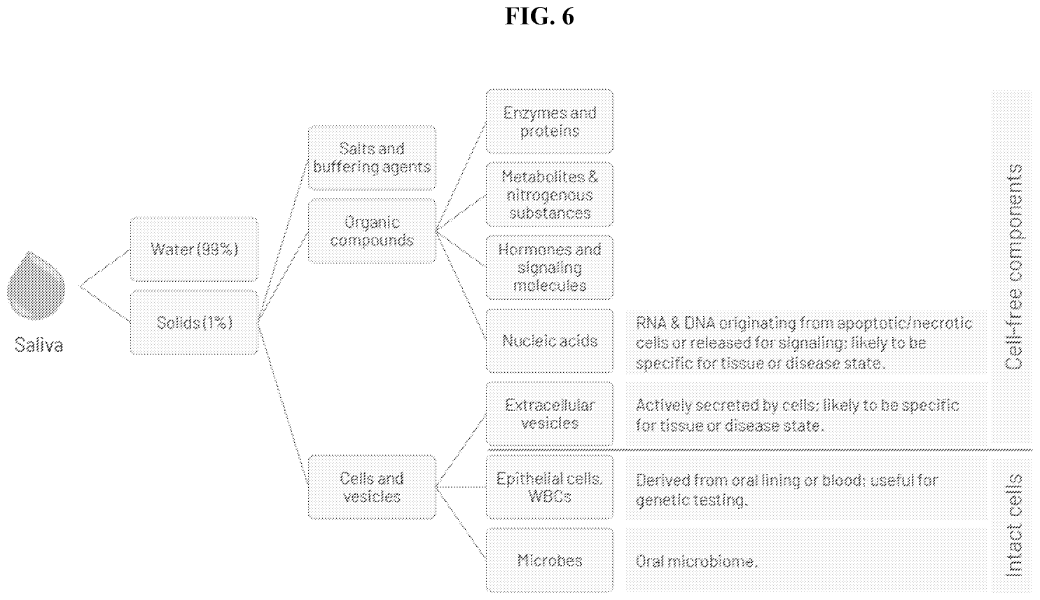

Described herein, in some aspects, is a method for detecting a disease or condition in a subject in need thereof. In some aspects, the method comprises analyzing a sample obtained from a subject for detecting a disease or condition in the subject. In some aspects, the method comprises analyzing a sample obtained from a subject for detecting or determining a likelihood of the subject developing the disease or condition in the future. In some embodiments, the method comprises analyzing the sample for a disease or condition that originates from a location that is different from a location where the sample is obtained. For example, the method can detect or determine the likelihood of the subject having or developing breast cancer by analyzing a sample (e.g., saliva) that is obtained from a non-breast sample. In some embodiments, the method comprises analyzing nucleic acid, such as an RNA transcript, in a sample. In some embodiments, the method comprises preserving the nucleic acid in the sample. In some embodiments, the preservation of the nucleic acid in the sample comprises using at least one denaturant to inactivate nucleases or filtration by at least one filter or a combination thereof. FIGS. 1-3 illustrate exemplary workflows utilizing the method described herein for analyzing the sample for detecting the disease or condition in the subject. In some embodiments, the method comprises determining over-expression or under-expression of transcripts in the sample, where the over or under expression of the transcript in the sample can correspond to over or under expression of the same transcript in a bodily location that is different from the collection location of the sample. For example, FIG. 4 and FIG. 5 illustrate that the transcripts found in saliva overlap significantly with those found in blood and esophagus mucosa tissue.

In some embodiments, saliva (also referred to as spit) is an extracellular fluid produced and secreted by salivary glands in the mouth. In some embodiments, in humans, saliva comprises water and solids. In some embodiments, the solids may comprise salts and buffering agents. In some embodiments, the solids may also comprise organic compounds. In some embodiments, the organic compounds may comprise enzymes and proteins. In some embodiments, the organic compounds may also comprise metabolites and nitrogenous substances. In some embodiments, the organic compounds may also comprise hormones and signaling molecules. In some embodiments, the organic compounds may also comprise nucleic acids. In some embodiments, the nucleic acids may comprise RNA or DNA. In some embodiments, the RNA or DNA may originate from apoptotic/necrotic cells or was released for signaling. In some embodiments, the solids may also comprise cells and vesicles. In some embodiments, the cells and vesicles may comprise extracellular vesicles. In some embodiments, the extracellular vesicles may be actively secreted by cells. In some embodiments, the cells and vesicles may also comprise or be derived from epithelial cells or white blood cells. In some embodiments, the epithelial cells or white blood cells are derived from oral lining or blood. In some embodiments, the cells and vesicles may also comprise or be derived from microbes. In some embodiments, the microbes may comprise cells of the oral microbiome.

In some embodiments, saliva may comprise cell-free components and intact cells. In some embodiments, the cell-free components may comprise salts and buffering agents, organic compounds, and some cells and vesicles in saliva. In some embodiments, the cell-free components may comprise enzymes and proteins. In some embodiments, the cell-free components may also comprise metabolites and nitrogenous substances. In some embodiments, the cell-free components may also comprise hormones and signaling molecules. In some embodiments, the cell-free components may also comprise nucleic acids. In some embodiments, the cell-free components may also comprise extracellular vesicles.

In some embodiments, the intact cells may comprise epithelial cells or white blood cells. In some embodiments, the intact cells may also comprise microbes.

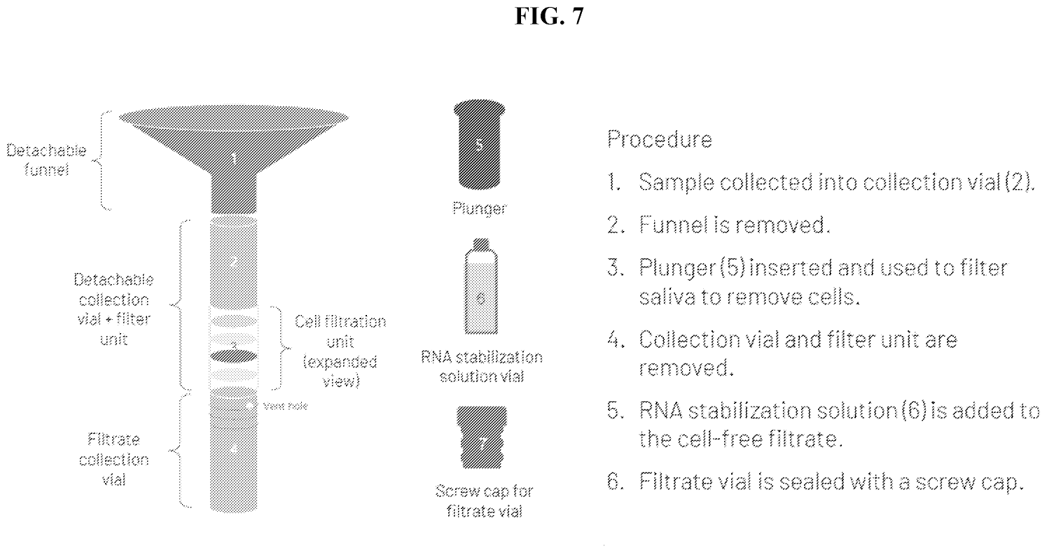

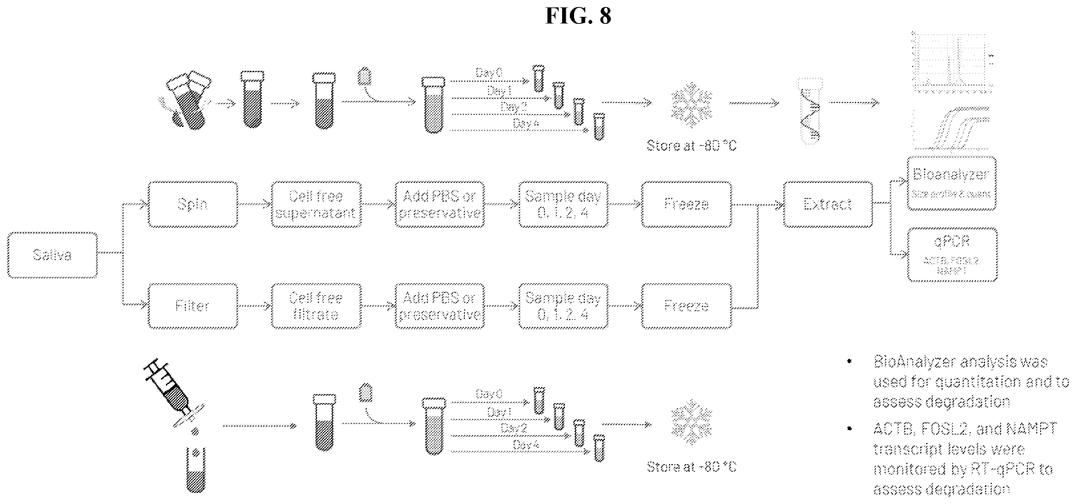

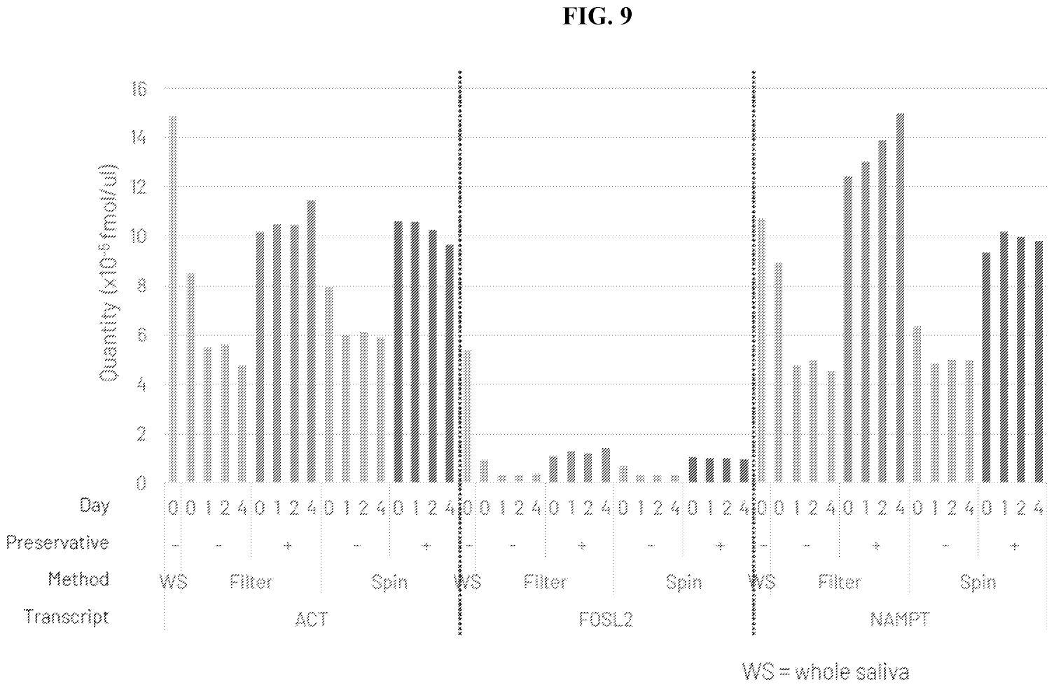

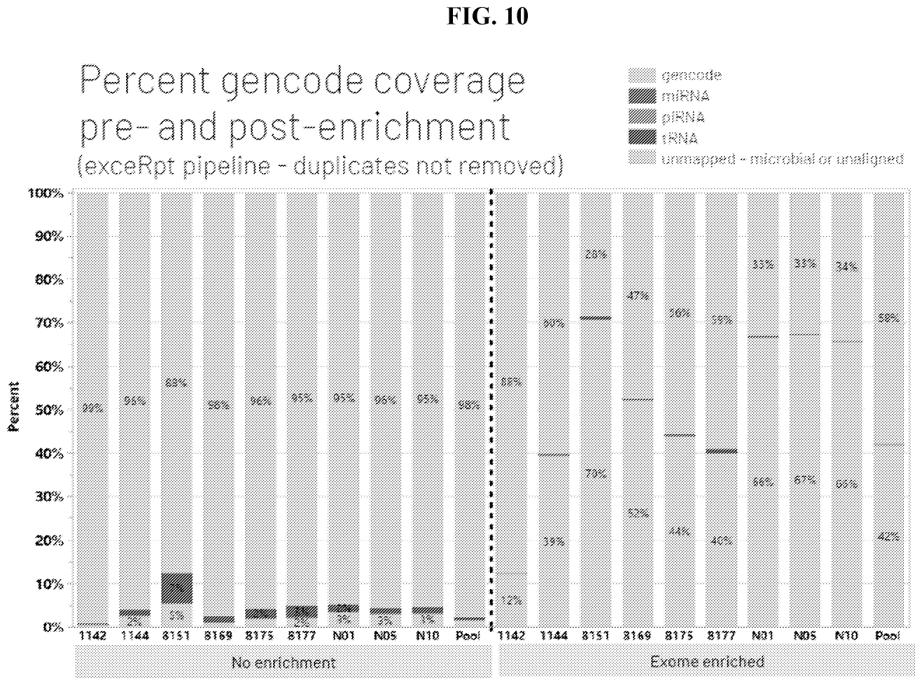

In some embodiments, the method comprises preserving nucleic acid in a sample obtained from the subject. FIGS. 7 and 8 illustrate various combinations of treatment of fractionation (e.g., by centrifugation and/or filtration), preservation using a denaturant, and quality control experiment for preserving and determining the integrity of the nucleic acid in the sample. FIGS. 8 and 9 illustrate experiments measuring the integrity of the nucleic acid preserved by the method described herein. FIG. 10 illustrates a chart comparing a percent GENCODE coverage of pre- and post-enrichment.

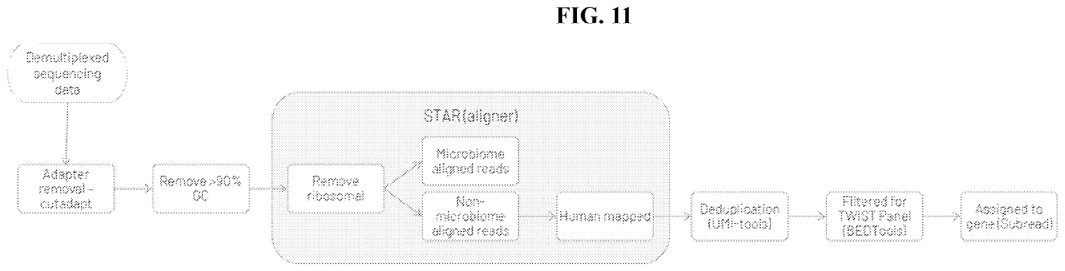

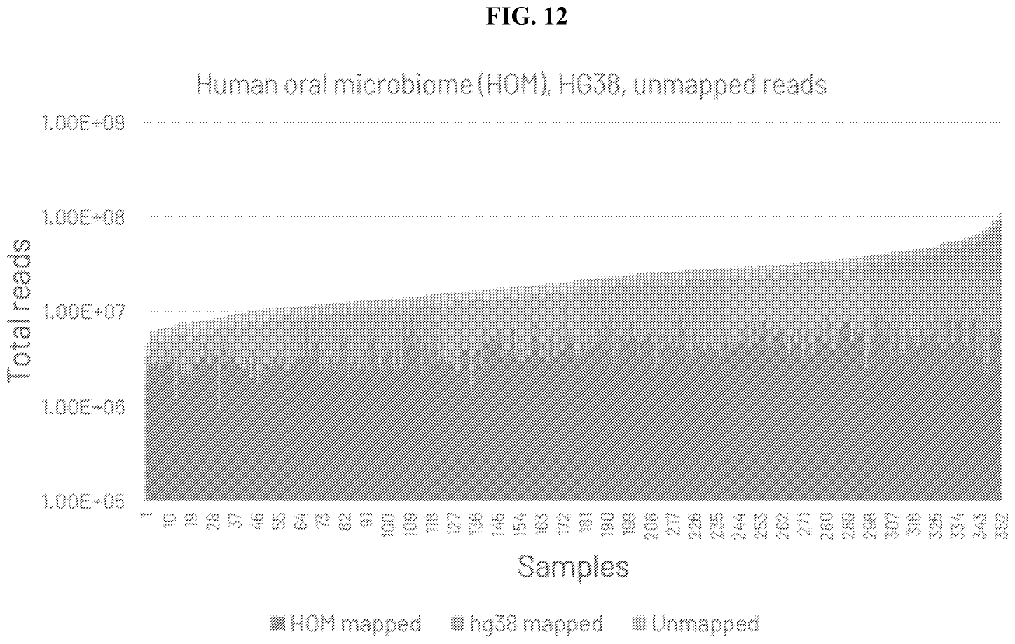

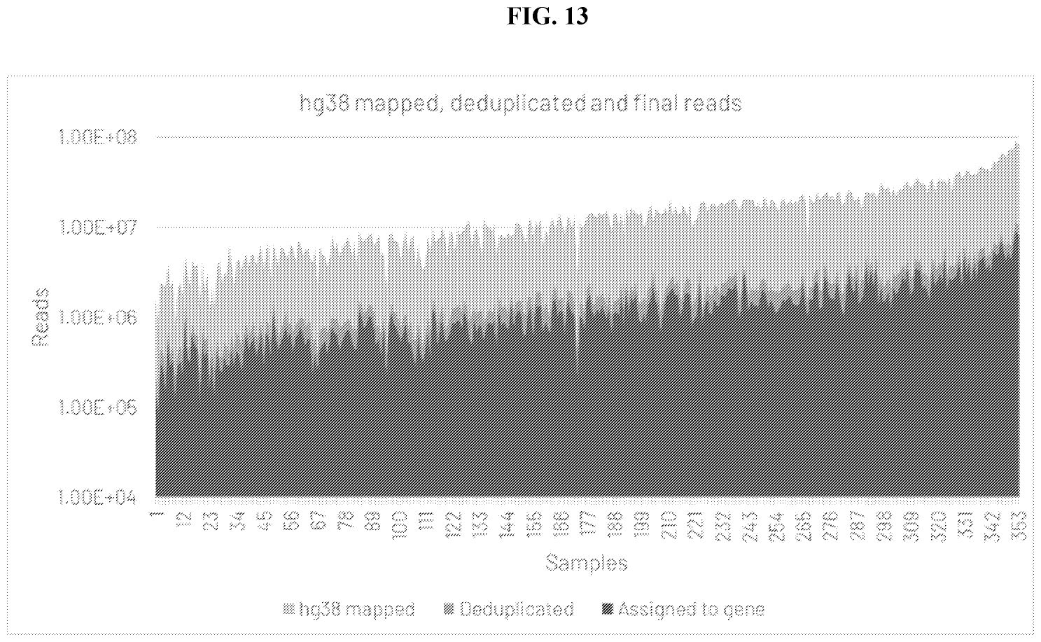

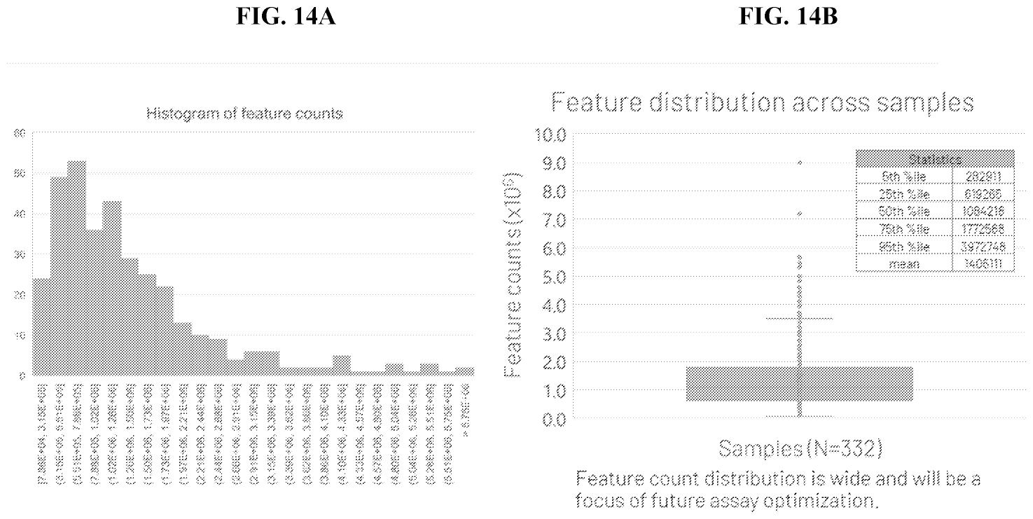

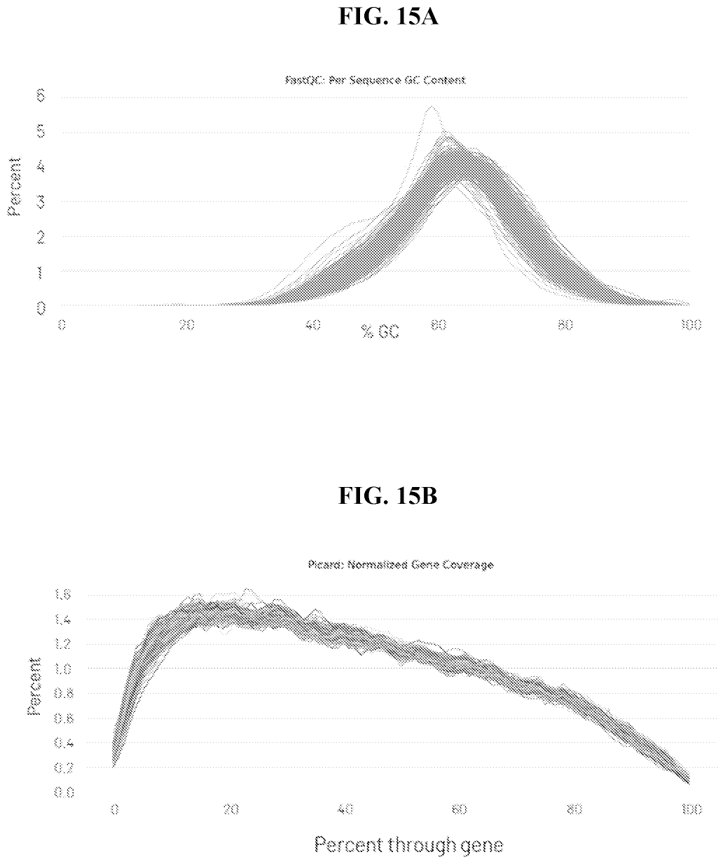

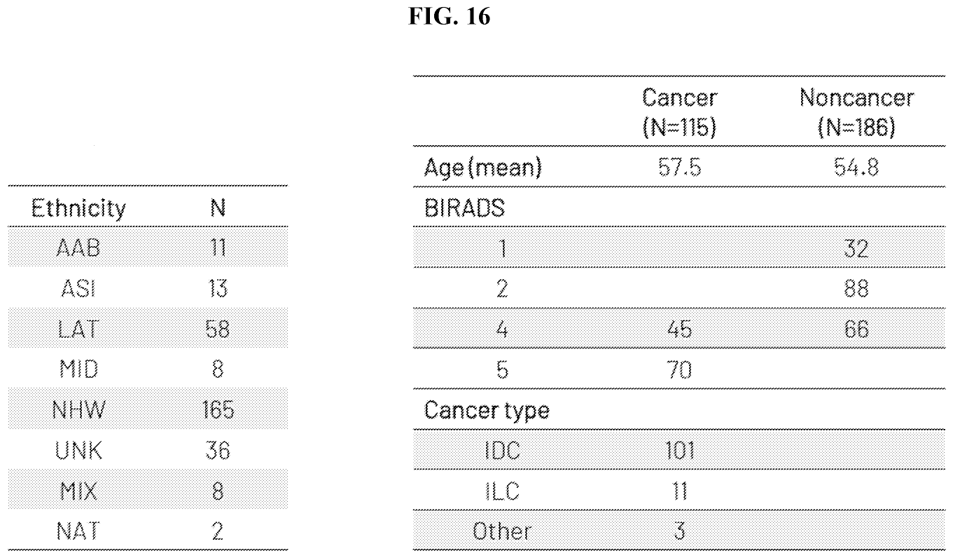

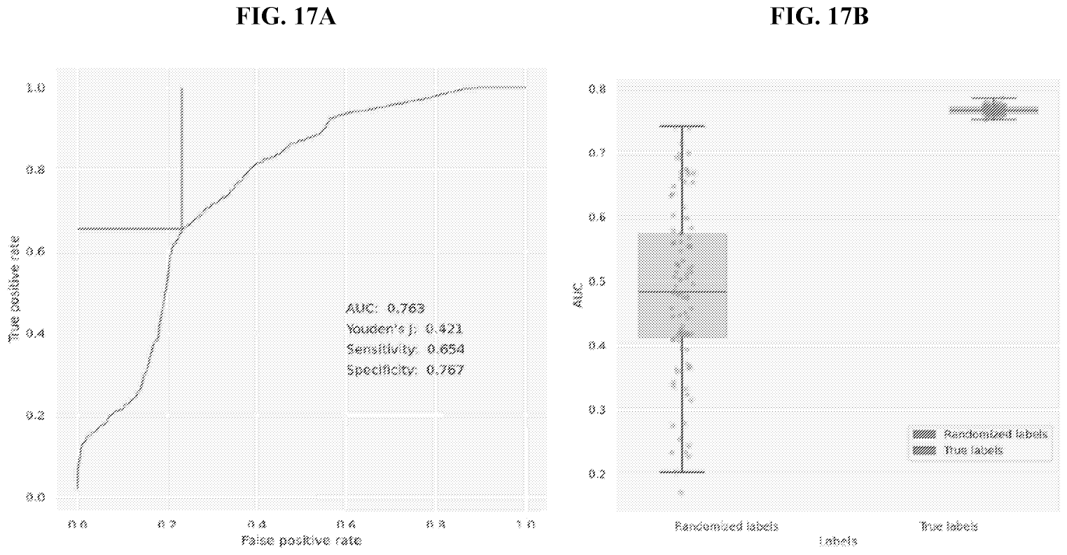

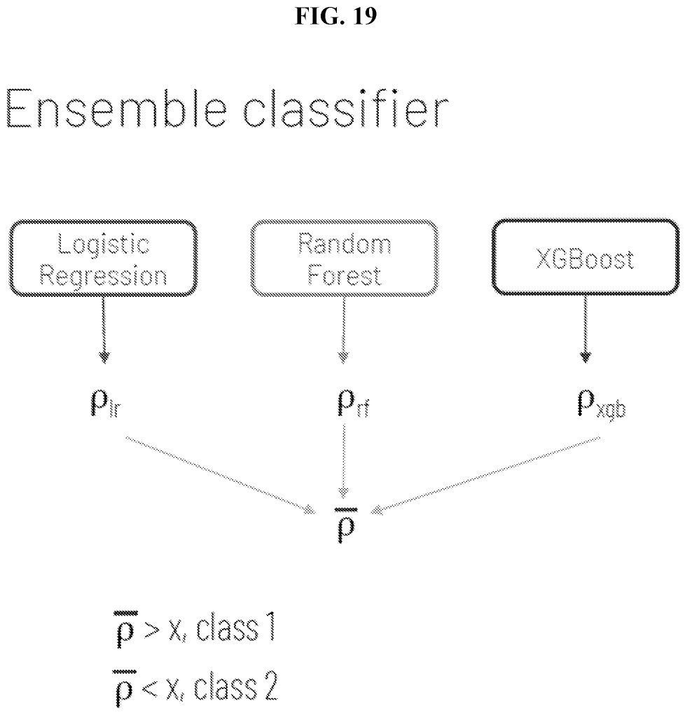

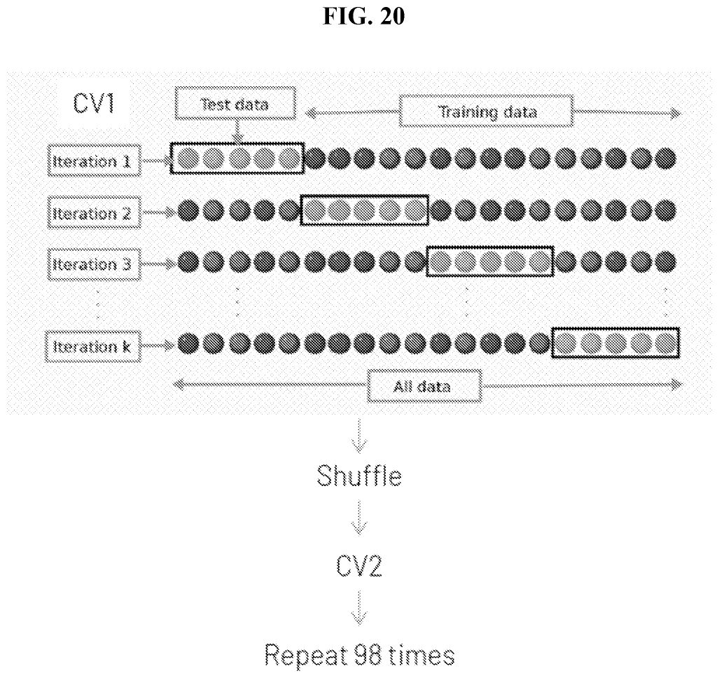

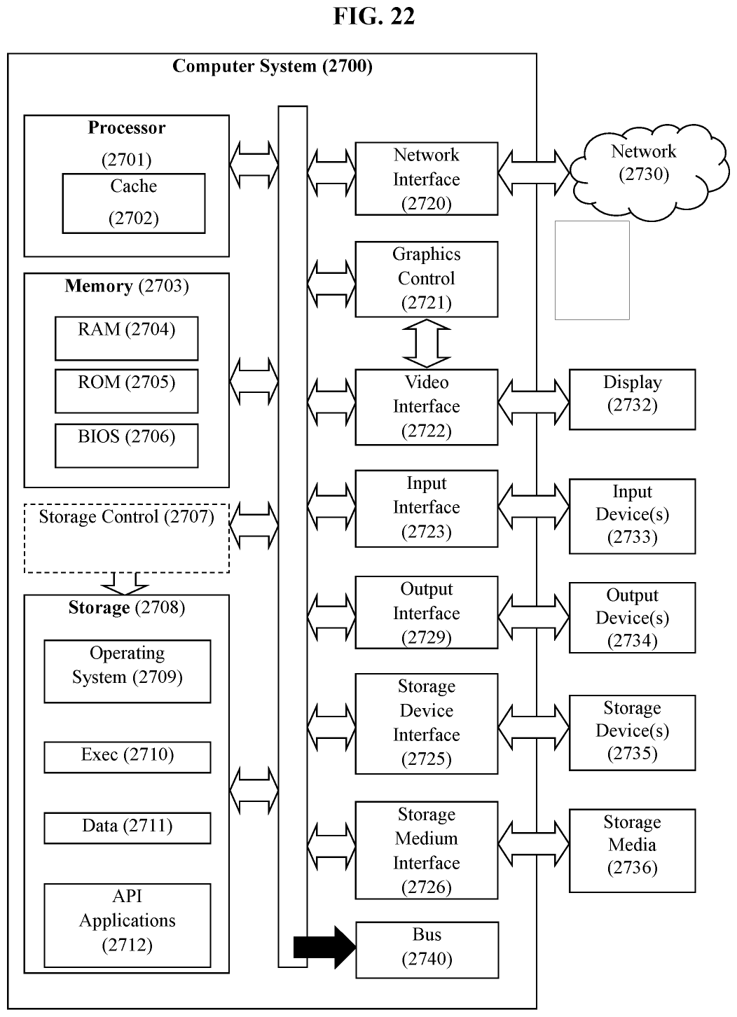

In some aspects, described herein is a method for analyzing the sample. In some embodiments, the method comprises using computer-implemented methods or machine learning based algorithms for analyzing, training, and refining the method of detection of the disease or condition described herein (e.g., FIGS. 11-20). FIGS. 16, 17A, 17B, 21A, 21B, and 21C illustrate examples of utilizing the method described herein for classifying clinical samples. FIGS. 22-24 illustrate non-limiting examples of computing device, application, or system for utilizing the method described herein. In some embodiments, the method increases sensitivity for detecting the disease or condition in the sample. In some embodiments, the method increases specificity for detecting the disease or condition in the sample. In some embodiments, the method decreases false positive for detecting the disease or condition in the sample. In some embodiments, the method decreases false negative for not detecting the disease or condition in the sample. In some embodiments, the method comprises generating a classifier based on the over-expression or under-expression of the transcripts detected in the sample. In some embodiments, the disease or condition is cancer. In some embodiments, the disease or condition is the likelihood of developing cancer.

Described herein, in some aspects, is a method for detecting the presence of a medical condition or disease for a subject, the method comprising: (a) optionally collecting a bodily fluid sample from a subject; (b) optionally, preserving the sample at the time of collection by addition of a preservative; (c) optionally, fractionating the sample; (d) optionally, adding a preservative to the fractionated sample; (e) selecting one or more analytes in the sample including, but are not limited to, nucleic acid transcripts or genomic regions of interest; (f) qualitatively or quantitatively detecting the selected analytes with an assay(s), wherein the assay(s) may include techniques involving, but are not limited to, biomolecule purification, biomolecule enrichment, biomolecule sequencing, PCR, quantitative PCR, isothermal amplification, mass spectrometry, antibody-based detection, or CRISPR and CRISPR-Cas systems, there by generating data; and (g) using a computer to analyze the data to detect the presence or absence of the medical condition or disease or analyzing the data to generate a likelihood score for the medical condition or disease.

In some embodiments, a sample comprises a bodily fluid sample, also referred to as a biofluid. In some embodiments, the bodily fluid sample comprises a saliva sample.

In some embodiments, at least one analyte comprises a nucleic acid. In some embodiments, at least one analyte comprises a cell-free RNA. In some embodiments, the nucleic acid comprises mRNA, small RNA, miRNA, snoRNA, snRNA, rRNAs, tRNA, siRNA, hnRNA, long non-coding RNA, shRNA, fragments thereof, or a combination thereof. In some embodiments, the nucleic acid comprises mRNA. In some embodiments, the nucleic acid comprises small RNA. In some embodiments, the nucleic acid comprises miRNA. In some embodiments, the nucleic acid comprises snoRNA. In some embodiments, the nucleic acid comprises snRNA. In some embodiments, the nucleic acid comprises rRNAs. In some embodiments, the nucleic acid comprises tRNA. In some embodiments, the nucleic acid comprises siRNA. In some embodiments, the nucleic acid comprises hnRNA. In some embodiments, the nucleic acid comprises long non-coding RNA. In some embodiments, the nucleic acid comprises shRNA. Fragments of any such nucleic acids are also contemplated.

In some embodiments, at least one analyte comprises a polypeptide. In some embodiments, the polypeptide is a protein. In some embodiments, the polypeptide is a metabolite.

In some embodiments, at least one analyte comprises a small molecule.

In some embodiments, at least one analyte comprises a cell.

Disclosed herein are also methods for stabilizing nucleic acid in a sample in which one or more protein denaturants is added to the sample to inhibit nuclease activity. In some embodiments, the nucleic acid is RNA. In some embodiments, the nucleic acid is DNA. In some embodiments, the nucleic acid is purified.

In some embodiments, one or more denaturant contains one or more chaotropic agents. In some embodiments, one or more chaotropic agents comprise detergent, urea, thiourea, guanidine thiocyanate, dodecylguanidine, dodine, or guanidine hydrochloride. In some embodiments, one or more chaotropic agents comprise detergent. In some embodiments, one or more chaotropic agents comprise urea. In some embodiments, one or more chaotropic agents comprise thiourea. In some embodiments, one or more chaotropic agents comprise guanidine thiocyanate. In some embodiments, one or more chaotropic agents comprise guanidine hydrochloride.

In some embodiments, one or more denaturants comprise guanidine thiocyanate at a concentration of between about 30% to about 70%. In some embodiments, one or more denaturants comprise guanidine thiocyanate at a concentration of between about 40% to about 70%. In some embodiments, one or more denaturants comprise guanidine thiocyanate at a concentration of between about 50% to about 70%. In some embodiments, one or more denaturants comprise guanidine thiocyanate at a concentration of between about 60% to about 70%. In some embodiments, one or more denaturants comprise guanidine thiocyanate at a concentration of between about 30% to about 60%. In some embodiments, one or more denaturants comprise guanidine thiocyanate at a concentration of between about 30% to about 50%. In some embodiments, one or more denaturants comprise guanidine thiocyanate at a concentration of between about 30% to about 40%.

In some embodiments, one or more denaturants comprise guanidine thiocyanate at a concentration of about 30%. In some embodiments, one or more denaturants comprise guanidine thiocyanate at a concentration of about 35%. In some embodiments, one or more denaturants comprise guanidine thiocyanate at a concentration of about 40%. In some embodiments, one or more denaturants comprise guanidine thiocyanate at a concentration of about 45%. In some embodiments, one or more denaturants comprise guanidine thiocyanate at a concentration of about 50%. In some embodiments, one or more denaturants comprise guanidine thiocyanate at a concentration of about 55%. In some embodiments, one or more denaturants comprise guanidine thiocyanate at a concentration of about 60%. In some embodiments, one or more denaturants comprise guanidine thiocyanate at a concentration of about 65%. In some embodiments, one or more denaturants comprise guanidine thiocyanate at a concentration of about 70%.

In some embodiments, a sample is a biofluid. In some embodiments, the biofluid comprises blood, serum, plasma, saliva, urine, sweat, tears, breast milk, colostrum, semen, or cerebrospinal fluid.

In some embodiments, a sample is filtered prior to addition of the one or more protein denaturants.

In some embodiments, a sample is collected in or transferred to a device with a filtration unit comprising at least one filter. In some embodiments, the device comprises a prefiltration mechanism to prevent, reduce, or inhibit clogging of smaller pore size filters; one or more filters; multiple filters arranged in order by pore size to allow successively smaller species to pass through; or at least two filters of the same type; a mechanism for applying mechanical force, centrifugal force, vacuum, capillary action, or radial or axial flow for filtering the sample through at least one filter. In some embodiments, the filtration is achieved using depth filtration. In some embodiments, the mechanism further comprises using a filtrate collection vessel prefilled with the one or more denaturants to allow for rapid nuclease inactivation upon contact with filtrate. In some embodiments, the filtered sample is removed or decanted from the device into a second vessel. In some embodiments, the filtered sample is removed or decanted from the device into a second vessel containing a preservative. In some embodiments, a collection unit or a filtration unit is separated from the filtrate collection vessel post-filtration to allow addition of the preservative. In some embodiments, the preservative is stored in an enclosure within a cap and released upon securing the cap onto the detached filtrate collection vessel.

In some embodiments, the nucleic acid is stabilized at a temperature range of between about −20° C. and about 50° C. In some embodiments, the nucleic acid is stabilized at a temperature range of between about −10° C. and about 50° C. In some embodiments, the nucleic acid is stabilized at a temperature range of between about 0° C. and about 50° C. In some embodiments, the nucleic acid is stabilized at a temperature range of between about 10° C. and about 50° C. In some embodiments, the nucleic acid is stabilized at a temperature range of between about 20° C. and about 50° C. In some embodiments, the nucleic acid is stabilized at a temperature range of between about 30° C. and about 50° C. In some embodiments, the nucleic acid is stabilized at a temperature range of between about −20° C. and about 40° C. In some embodiments, the nucleic acid is stabilized at a temperature range of between about −20° C. and about 30° C. In some embodiments, the nucleic acid is stabilized at a temperature range of between about −20° C. and about 20° C. In some embodiments, the nucleic acid is stabilized at a temperature range of between about −20° C. and about 10° C. In some embodiments, the nucleic acid is stabilized at a temperature range of between about −20° C. and about 0° C. In some embodiments, the nucleic acid is stabilized at a temperature range of between about −20° C. and about −10° C.

In some embodiments, the nucleic acid is stabilized at about −20° C. In some embodiments, the nucleic acid is stabilized at about −15° C. In some embodiments, the nucleic acid is stabilized at about −10° C. In some embodiments, the nucleic acid is stabilized at about −5° C. In some embodiments, the nucleic acid is stabilized at about 0° C. In some embodiments, the nucleic acid is stabilized at about 5° C. In some embodiments, the nucleic acid is stabilized at about 10° C. In some embodiments, the nucleic acid is stabilized at about 15° C. In some embodiments, the nucleic acid is stabilized at about 20° C. In some embodiments, the nucleic acid is stabilized at about 25° C. In some embodiments, the nucleic acid is stabilized at about 30° C. In some embodiments, the nucleic acid is stabilized at about 35° C. In some embodiments, the nucleic acid is stabilized at about 40° C. In some embodiments, the nucleic acid is stabilized at about 45° C. In some embodiments, the nucleic acid is stabilized at about 50° C.

In some embodiments, the nucleic acid is stabilized for at last 5 days.

In some embodiments, the nucleic acid is stabilized for at last 1 days. In some embodiments, the nucleic acid is stabilized for at last 2 days. In some embodiments, the nucleic acid is stabilized for at last 3 days. In some embodiments, the nucleic acid is stabilized for at last 4 days. In some embodiments, the nucleic acid is stabilized for at least 5 days.

In some embodiments, at least one filter comprises low nucleic acid-binding material.

In some embodiments, at least one filter has a size cutoff at least about 0.1 μm, at least about 0.5 μm, at least about 1 μm, at least about 5 μm, at least about 10 μm, at least about 15 μm, at least about 20 μm, or at least about 25 μm. In some embodiments, at least one filter has a size cutoff at least about 0.1 μm. In some embodiments, at least one filter has a size cutoff at least about 0.5 μm. In some embodiments, at least one filter has a size cutoff at least about 1 μm. In some embodiments, at least one filter has a size cutoff at least about 5 μm. In some embodiments, at least one filter has a size cutoff at least about 10 μm. In some embodiments, at least one filter has a size cutoff at least about 15 μm. In some embodiments, at least one filter has a size cutoff at least about 20 μm. In some embodiments, at least one filter has a size cutoff at least about 25 μm.

In some embodiments, at least one filter has a size cutoff at most about 0.1 μm, at most about 0.5 μm, at most about 1 μm, at most about 5 μm, at most about 10 μm, at most about 15 μm, at most about 20 μm, or at most about 25 μm. In some embodiments, at least one filter has a size cutoff at most about 0.1 μm. In some embodiments, at least one filter has a size cutoff at most about 0.5 μm. In some embodiments, at least one filter has a size cutoff at most about 1 μm. In some embodiments, at least one filter has a size cutoff at most about 5 μm. In some embodiments, at least one filter has a size cutoff at most about 10 μm. In some embodiments, at least one filter has a size cutoff at most about 15 μm. In some embodiments, at least one filter has a size cutoff at most about 20 μm. In some embodiments, at least one filter has a size cutoff at most about 25 μm.

In some embodiments, at least one filter has a size cutoff between about 0.1 μm and about 25 μm. In some embodiments, at least one filter has a size cutoff between about 0.1 μm and about 20 μm. In some embodiments, at least one filter has a size cutoff between about 0.1 μm and about 15 μm. In some embodiments, at least one filter has a size cutoff between about 0.1 μm and about 10 μm. In some embodiments, at least one filter has a size cutoff between about 0.1 μm and about 9 μm. In some embodiments, at least one filter has a size cutoff between about 0.1 μm and about 8 μm. In some embodiments, at least one filter has a size cutoff between about 0.1 μm and about 7 μm. In some embodiments, at least one filter has a size cutoff between about 0.1 μm and about 6 μm. In some embodiments, at least one filter has a size cutoff between about 0.1 μm and about 5 μm. In some embodiments, at least one filter has a size cutoff between about 0.1 μm and about 4 μm. In some embodiments, at least one filter has a size cutoff between about 0.1 μm and about 3 μm. In some embodiments, at least one filter has a size cutoff between about 0.1 μm and about 2 μm. In some embodiments, at least one filter has a size cutoff between about 0.1 μm and about 1 μm. In some embodiments, at least one filter has a size cutoff between about 0.1 μm and about 0.5 μm. In some embodiments, at least one filter has a size cutoff between about 0.5 μm and about 25 μm. In some embodiments, at least one filter has a size cutoff between about 1 μm and about 25 μm. In some embodiments, at least one filter has a size cutoff between about 2 μm and about 25 μm. In some embodiments, at least one filter has a size cutoff between about 3 μm and about 25 μm. In some embodiments, at least one filter has a size cutoff between about 4 μm and about 25 μm. In some embodiments, at least one filter has a size cutoff between about 5 μm and about 25 μm. In some embodiments, at least one filter has a size cutoff between about 6 μm and about 25 μm. In some embodiments, at least one filter has a size cutoff between about 7 μm and about 25 μm. In some embodiments, at least one filter has a size cutoff between about 8 μm and about 25 μm. In some embodiments, at least one filter has a size cutoff between about 9 μm and about 25 μm. In some embodiments, at least one filter has a size cutoff between about 10 μm and about 25 μm. In some embodiments, at least one filter has a size cutoff between about 15 μm and about 25 μm. In some embodiments, at least one filter has a size cutoff between about 20 μm and about 25 μm.

In some embodiments, at least one filter has a size cutoff about 0.1 μm. In some embodiments, at least one filter has a size cutoff about 0.5 μm. In some embodiments, at least one filter has a size cutoff about 1 μm. In some embodiments, at least one filter has a size cutoff about 2 μm. In some embodiments, at least one filter has a size cutoff about 3 μm. In some embodiments, at least one filter has a size cutoff about 4 μm. In some embodiments, at least one filter has a size cutoff about 5 μm. In some embodiments, at least one filter has a size cutoff about 6 μm. In some embodiments, at least one filter has a size cutoff about 7 μm. In some embodiments, at least one filter has a size cutoff about 8 μm. In some embodiments, at least one filter has a size cutoff about 9 μm. In some embodiments, at least one filter has a size cutoff about 10 μm. In some embodiments, at least one filter has a size cutoff about 12 μm. In some embodiments, at least one filter has a size cutoff about 14 μm. In some embodiments, at least one filter has a size cutoff about 16 μm. In some embodiments, at least one filter has a size cutoff about 18 μm. In some embodiments, at least one filter has a size cutoff about 20 μm. In some embodiments, at least one filter has a size cutoff about 22 μm. In some embodiments, at least one filter has a size cutoff about 24 μm. In some embodiments, at least one filter has a size cutoff about 25 μm.

In some embodiments, a filter retains a plurality of white blood cells. In some embodiments, a filter retains a plurality of red blood cells. In some embodiments, a filter retains a plurality of cells derived from solid tissues. In some embodiments, a filter retains a plurality of microbes.

In some embodiments, a filter comprises synthetic material to minimize the introduction of contaminating nucleic acid. In some embodiments, a filter comprises biological material.

In some embodiments, the filtering produces a cell-free biofluid or a cell-depleted biofluid. In some embodiments, the filtering produces cell-free plasma or cell-depleted plasma. In some embodiments, the filtering produces cell-free saliva or cell-depleted saliva. In some embodiments, the filtering produces cell-free urine or cell-depleted urine.

In some embodiments, the disease or condition (or medical condition) is a cancer. In some embodiments, the cancer type is a solid cancer type or a hematologic malignant cancer type. In some embodiments, the cancer type is a metastatic cancer type or a relapsed or refractory cancer type. In some embodiments, the cancer type comprises acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblstoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, lip cancer, liver cancer, Lymphoid Neoplasm Diffuse Large B-cell Lymphoma [DLBCL), malignant fibrous histiocytoma bone cancer, medulloblastoma, medullo epithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, or Wilm's tumor. In some embodiments, the cancer type comprises acute lymphoblastic leukemia, acute myeloid leukemia, bladder cancer, breast cancer, brain cancer, cervical cancer, cholangiocarcinoma (CHOL), colon cancer, colorectal cancer, endometrial cancer, esophagus cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, pheochromocytoma and paraganglioma (PCPG), prostate cancer, rectum cancer, sarcoma, skin cancer, squamous cell carcinoma, testicular cancer, stomach cancer, or thyroid cancer. In some embodiments, the cancer type comprises bladder cancer, breast cancer, cervical cancer, cholangiocarcinoma (CHOL), colon cancer, esophagus cancer, head and neck cancer, kidney cancer, liver cancer, lung cancer, pancreatic cancer, pheochromocytoma and paraganglioma (PCPG), prostate cancer, rectum cancer, sarcoma, skin cancer, stomach cancer, or thyroid cancer.

In some embodiments, the disease or condition is a breast cancer. In some embodiments, the cancer is a lung cancer, an esophageal cancer, or a head and neck cancer.

In some embodiments, a disease or condition is a neurological disease. In some embodiments, a disease or condition is an autoimmune disease. In some embodiments, a disease or condition is a metabolic disease. In some embodiments, a disease or condition is an endocrine disease. In some embodiments, a disease or condition is a digestive tract disease.

In some embodiments, a disease or condition is an injury.

In some embodiments, a disease or condition is pregnancy.

### Nucleic Acid Preservation

Described herein, in some aspects, is a method for preservation of nucleic acids in which the solution containing the nucleic acids is protected from degradation for extended periods by a preservative, without the need for refrigeration. Biological solutions may be filtered prior to preservative addition. Filtration is particularly advantageous in the case of biofluids if the cells can be separated away to leave primarily the cell-free nucleic acids for analysis. Nucleases present in biological samples are a primary root cause of nucleic acid degradation. Their action can be inhibited by the addition of nuclease inhibitors as described below.

In some aspects, metal chelators, such as ethylenediaminetetraacetic acid (EDTA), can inhibit nucleases that are dependent on divalent cations for their activity. However, in some aspects, this method does not inhibit all nucleases because there are some nucleases that do not require divalent ions.

In some aspects, competitive inhibitors which bind to an enzyme active site may also be used, but because there are many different types of nucleases, a multitude of competitive inhibitors is required, and it is difficult to universally suppress nucleolytic activity with this approach.

An alternative strategy to prevent nucleolytic degradation is to destroy the nuclease by one of three means: (i) heat denaturation of nucleases; (ii) digestion of nucleases by proteases; and (iii) chemical denaturation of nucleases.

In some aspects, the method of heat denaturation of nucleases has limitations in that some nucleases may renature to a native conformation upon cooling and that the treatment itself can damage nucleic acids. This method can also be slow, particularly, for larger sample volumes, and nucleases may have an opportunity to substantially degrade nucleic acids before they are inactivated.

In some aspects, digestion of nucleases by proteases can be effective in degrading nucleases, but because of its inherent slow process, nucleases may have an opportunity to substantially degrade nucleic acids before they are fully digested.

In some aspects, chemical denaturation of nucleases, such as addition of an agent that denatures proteins, can render nucleases inactive. This strategy has the advantage that nuclease activity can be completely inhibited without compromising the nucleic acids until such time that the nucleic acids can be extracted.

In some embodiments, the preserving the sample comprises contacting the sample with a preservative. In some embodiments, the preservative comprises at least one of the following: ethylenediaminetetraacetic acid (EDTA); an RNase inhibitor; an anti-microbial; a denaturing agent; an agent that inhibits nuclease activity; a sequestration agent; a buffering agent; a salt; an osmolyte; or a combination thereof. In some embodiments, the preservative comprises ethylenediaminetetraacetic acid (EDTA). In some embodiments, the preservative comprises an Rnase inhibitor. In some embodiments, the preservative comprises an anti-microbial. In some embodiments, the preservative comprises a denaturing agent. In some embodiments, the preservative comprises an agent that inhibits nuclease activity. In some embodiments, the preservative comprises a sequestration agent. In some embodiments, the preservative comprises a buffering agent. In some embodiments, the preservative comprises a salt. In some embodiments, the preservative comprises an osmolyte.

In some embodiments, the denaturing agent comprises a nucleic acid denaturing agent or a protein denaturing agent. The denaturing agent comprises a nucleic acid denaturing agent. The denaturing agent comprises a protein denaturing agent.

### Device

Also described herein is a device, an example of which is shown in FIG. 7. The device is for collecting and stabilizing an analyte in a sample and the device comprises a) a sample collection vessel 2 for collecting the sample; b) a filtration unit 3 in fluid communication with the sample collection vessel 2, the filtration unit 3 comprising at least one filter for filtering the sample to produce a filtrate; and c) a filtrate collection vessel 4 in fluid communication with the filtration unit 3 for collecting the filtrate and contacting the filtrate with a preservative.

In embodiments, the filtration unit 2 comprises multiple filters arranged in order by pore size to allow successively smaller species to pass through. In additional or alternative embodiments, the filtration unit 2 comprises at least two filters of the same type. In embodiments, the device comprises a prefiltration mechanism to prevent clogging of smaller pore size filters. For example, the filtration unit 2 may comprise the prefiltration mechanism or the prefiltration mechanism may be separate from the cell filtration unit. In some embodiments, the filtration unit comprises a single filter, two filters, three filters, four filters, five filters, six filters, seven filters, eight filters, nine filters, 10 filters, or more than 10 filters.

The filters may be of any know type that would be suitable for filtering a biofluid sample. For example, in embodiments, the at least one filter comprises a depth filter, an asymmetric filter, a microporous filter, or a combination thereof. In embodiments, the at least one filter comprises low nucleic acid-binding material.

In embodiments, the at least one filter may have a size cutoff that is selected to exclude or let pass through any desired components based on size. For example, in embodiments, the at least one filter has a size cutoff of at least about 0.1 μm, at least about 1 μm, at least about 2 μm, at least about 3 μm, at least about 4 μm, at least about 5 μm, at least about 10 μm, at least about 15 μm, at least about 20 μm, at least about 25 μm, at least about 30, at least about 35 μm, at least about 40 μm, at least about 45 μm, at least about 50 μm, at least about 55 μm, at least about 60 μm, at least about 65 μm, at least about 70 μm, at least about 75 μm, at least about 80 μm, at least about 85 μm, at least about 90 μm, at least about 95 μm, or at least about 100 μm. In additional or alternative embodiments, the at least one filter has a size cutoff at most about 0.1 μm, at most about 1 μm, at most about 2 μm, at most about 3 μm, at most about 4 μm, at most about 5 μm, at most about 10 μm, at most about 15 μm, at most about 20 μm, at most about 25 μm, at most about 30, at most about 35 μm, at most about 40 μm, at most about 45 μm, at most about 50 μm, at most about 55 μm, at most about 60 μm, at most about 65 μm, at most about 70 μm, at most about 75 μm, at most about 80 μm, at most about 85 μm, at most about 90 μm, at most about 95 μm, or at most about 100 μm. For example, in embodiments, the at least one filter has a size cutoff between about 0.1 μm and about 100 μm.

The at least one filter may similarly have any desired thickness. For example, in embodiments, the filter has a thickness of from about 50 μm to about 1000 μm, or from about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, or about 950 μm to about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 100 μm, such as from about 355 μm to about 560 μm, such as about 330 μm, such as from about 120 μm to about 170 μm, such as from about 230 μm to about 270 μm, such as from about 480 μm to about 640 μm.

More than one filter can be stacked together, resulting in a stack height, which is the combined thicknesses of the multiple filters. In embodiments, the filtration unit 2 comprises a filter stack height of from about 120 μm to about 10000 μm, such as from about 120 μm, about 150 μm, about 175 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1250 μm, about 1500 μm, about 1750 μm, about 2000 μm, about 2500 μm, about 3000 μm, about 3500 μm, about 4000 μm, about 4500 μm, about 5000 μm, about 5500 μm, about 6000 μm, about 6500 μm, about 7000 μm, about 7500 μm, about 8000 μm, about 8500 μm, about 9000 μm, or about 9500 μm to about 150 μm, about 175 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1250 μm, about 1500 μm, about 1750 μm, about 2000 μm, about 2500 μm, about 3000 μm, about 3500 μm, about 4000 μm, about 4500 μm, about 5000 μm, about 5500 μm, about 6000 μm, about 6500 μm, about 7000 μm, about 7500 μm, about 8000 μm, about 8500 μm, about 9000 μm, about 9500 μm, or about 10000 μm.

Most filters are circular and have a diameter. While the filters can be of any shape, they are typically circular and have a diameter of from about 10 mm to about 50 mm, such as from about 10 mm, about 15 mm, about 20 mm, about 25 mm, about 30 mm, about 35 mm, about 40 mm, or about 45 mm to about 15 mm, about 20 mm, about 25 mm, about 30 mm, about 35 mm, about 40 mm, about 45 mm, or about 50 mm.

In embodiments, the at least one filter is hydrophilic or hydrophobic. In additional or alternative embodiments, the at least one filter comprises polysulfone and/or polypropylene. In some embodiments, the at least one filter comprises synthetic material to minimize the introduction of contaminating nucleic acid. In other embodiments, the at least one filter comprises biological material or is free of biological material, such as cellulose. In some embodiments, especially when isolating or stabilizing a low quantity analyte, contaminating components from biological materials, such as contaminating nucleic acids that might be found in cellulose or other biological materials, can confound the results. It is therefore advantageous in some embodiments to avoid the use of biological materials in the filter materials.

In embodiments, the at least one filter retains a plurality of white blood cells. In additional or alternative embodiments, the at least one filter retains a plurality of red blood cells. In additional or alternative embodiments, the at least one filter retains a plurality of cells derived from solid tissues. In additional or alternative embodiments, the at least one filter retains a plurality of microbes.

The sample is typically a biofluid. Biofluids can include any bodily fluid, examples of which comprise blood, serum, plasma, saliva, urine, sweat, tears, breast milk, colostrum, semen, or cerebrospinal fluid.

The filtrate collected in the device is, in embodiments, a cell-free biofluid or a cell-depleted biofluid. In embodiments, the filtrate is cell-free plasma or cell-depleted plasma. In embodiments, the filtrate is cell-free saliva or cell-depleted saliva. In embodiments, the filtrate is cell-free urine or cell-depleted urine.

In embodiments, the device further comprises a mechanism for applying mechanical force, centrifugal force, vacuum, capillary action, or radial or axial flow for filtering the sample through at least one of the at least one filter. In embodiments, the mechanism is for applying mechanical force, centrifugal force, vacuum, capillary action, or radial or axial flow for filtering the sample through all filters in the device. In some embodiments, the mechanism comprises a plunger 5 that engages with the sample collection vessel 2 to push the sample through the filtration unit 3 and into the filtrate collection vessel 4. In embodiments, the plunger 5 is integral with or separate from the sample collection vessel 2.

In embodiments, the sample collection vessel 2 comprises a funnel 1, wherein the funnel 1 is integral with or couplable to the sample collection vessel 2.

In some embodiments, the filtrate collection vessel 4 comprises the preservative 6. In other embodiments, the preservative 6 is provided separately, for example in its own vessel. It will be understood that the preservative may be added to the sample prior to putting the sample into the sample collection vessel 2, it may be added to the sample in the sample collection vessel 2, it may already be present in the filtrate collection vessel 4, or it may be separately added to the filtrate collection vessel 4 before, during, or after collecting the filtrate in the filtrate collection vessel. For example, in embodiments, the filtrate collection vessel 4 is detachable from the filtration unit 2. In embodiments, the device further comprises a cap 7 for the detached filtrate collection vessel 2. In some embodiments, the cap 7 comprises an enclosure storing the preservative 6 that is released upon securing the cap 7 onto the detached filtrate collection vessel 4. In additional or alternative embodiments, the device further comprises a second vessel for decanting the filtrate. In embodiments, the second vessel comprises the preservative 6.

Any preservative may be used as will be understood by the skilled person. In some embodiments, the preservative comprises at least one of the following: ethylenediaminetetraacetic acid (EDTA); an RNase inhibitor; an anti-microbial; a denaturing agent; an agent that inhibits nuclease activity, a sequestration agent; a buffering agent; a salt; an osmolyte; or a combination thereof. For example, in embodiments, the denaturing agent comprises a nucleic acid denaturing agent or a protein denaturing agent. In some embodiments, the agent that inhibits nuclease activity comprises one or more protein denaturants, EDTA, detergents such as SDS, aurintricarboxylic acid (ATA), chelating agents, or combinations thereof. In embodiments, the one or more protein denaturants comprise one or more chaotropic agents comprising detergent, urea, thiourea, guanidine thiocyanate, dodecylguanidine, dodine, or guanidine hydrochloride. In some embodiments, the one or more protein denaturants comprise the guanidine thiocyanate at a concentration of between about 30% and about 70%, such as from about 30%, about 35%, about 40%, about 45%, about 50%, about 55%, about 60%, or about 65% to about 35%, about 40%, about 45%, about 50%, about 55%, about 60%, about 65%, or about 70%.

In embodiments, the device stabilizes the analyte at a desired temperature range, such as at freezing temperatures, at refrigerator temperatures, at room temperature, at body temperature, or at higher than body temperatures. For example, in embodiments, the device stabilizes the analyte at temperatures of between about −20° C. and about 50° C., such as from about −20° C., about −10° C., about −5° C., about 0° C., about 2° C., about 4° C., about 8° C., about 10° C., about 15° C., about 20° C., about 30° C., about 37° C., or about 40° C. to about −10° C., about −5° C., about 0° C., about 2° C., about 4° C., about 8° C., about 10° C., about 15° C., about 20° C., about 30° C., about 37° C., about 40° C., or about 50° C.

In embodiments, the device stabilizes the analyte for at least 5 days, such as at least 1 day, at least 2 days, at least 3 days, at least 4 days, at least 5 days, at least 6 days, at least 7 days, at least 10 days, at least 2 weeks, at least one month, at least two months, at least 3 months, at least 6 months, at least a year, or more.

In embodiments, the at least one analyte comprises a cell-free analyte.

In embodiments, the at least one analyte comprises a nucleic acid. Any nucleic acid or fragments of nucleic acids are contemplated. For example, in embodiments, the nucleic acid comprises a cell-free RNA. In embodiments, the nucleic acid comprises mRNA, small RNA, miRNA, snoRNA, snRNA, IRNAs, tRNA, siRNA, hnRNA, long non-coding RNA, shRNA, fragments thereof, or a combination thereof.

In embodiments, the at least one analyte comprises a polypeptide. In embodiments, the polypeptide is a protein. In additional or alternative embodiments, the polypeptide is a metabolite.

In embodiments, the at least one analyte comprises a small molecule. In embodiments, the at least one analyte comprises a metabolite. In embodiments, the at least one analyte comprises a cell.

In embodiments, the filtration unit is detachable for retentate recovery so that biomolecules and cells contained within the retentate can be preserved and analyzed.

In some embodiments, the at least one filter reduces the viscosity of the filtrate as compared to the sample. For example, the at least one filter may remove components from the sample that contribute to viscosity, such as mucins.

In embodiments, the device described herein is provided as a kit. The kit in embodiments comprises the device including the sample collection vessel, the filtration unit, and the filtrate collection vessel along with at least one additional component, such as a plunger; a cap for the filtrate collection vessel; a funnel; a preservative; and/or instructions for use.

Also described herein is a method for stabilizing an analyte in a sample. The method comprises applying the sample to the sample collection vessel of the device or the kit described herein, filtering the sample through the filtration unit, and contacting the filtrate with the preservative.

When the above method is used to stabilize an analyte, a stabilized analyte is collected. Thus also described herein is an analyte stabilized by the method.

### Computing System

Referring to FIG. 22, a block diagram is shown depicting an exemplary machine that includes a computer system 2700 (e.g., a processing or computing system) within which a set of instructions can execute for causing a device to perform or execute any one or more of the aspects and/or methodologies for static code scheduling of the present disclosure. The components in FIG. 22 are examples only and do not limit the scope of use or functionality of any hardware, software, embedded logic component, or a combination of two or more such components implementing particular embodiments.

Computer system 2700 may include one or more processors 2701, a memory 2703, and a storage 2708 that communicate with each other, and with other components, via a bus 2740. The bus 2740 may also link a display 2732, one or more input devices 2733 (which may, for example, include a keypad, a keyboard, a mouse, a stylus, etc.), one or more output devices 2734, one or more storage devices 2735, and various tangible storage media 2736. All of these elements may interface directly or via one or more interfaces or adaptors to the bus 2740. For instance, the various tangible storage media 2736 can interface with the bus 2740 via storage medium interface 2726. Computer system 2700 may have any suitable physical form, including, but is not limited to, one or more integrated circuits (Ics), printed circuit boards (PCBs), mobile handheld devices (such as mobile telephones or PDAs), laptop or notebook computers, distributed computer systems, computing grids, or servers.

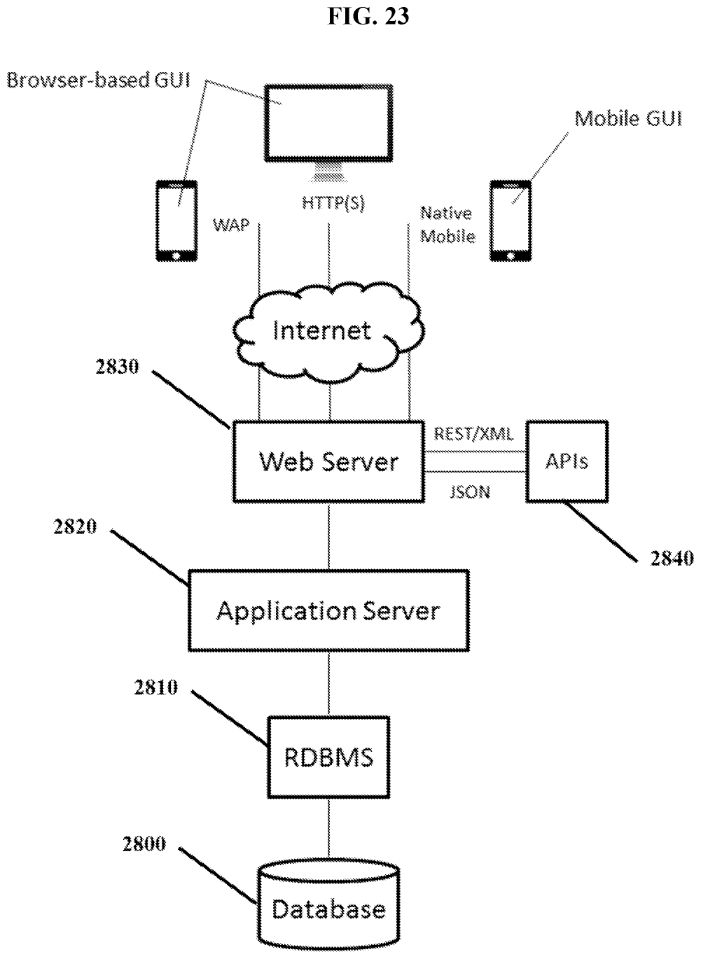

Computer system 2700 includes one or more processor(s) 2701 (e.g., central processing units (CPUs) or general-purpose graphics processing units (GPGPUs)) that carry out functions. Processor(s) 2701 optionally contains a cache memory unit 2702 for temporary local storage of instructions, data, or computer addresses. Processor(s) 2701 are configured to assist in execution of computer readable instructions. Computer system 2700 may provide functionality for the components depicted in FIG. 23 as a result of the processor(s) 2701 executing non-transitory, processor-executable instructions embodied in one or more tangible computer-readable storage media, such as memory 2703, storage 2708, storage devices 2735, and/or storage medium 2736. The computer-readable media may store software that implements particular embodiments, and processor(s) 2701 may execute the software. Memory 2703 may read the software from one or more other computer-readable media (such as mass storage device(s) 2735, 2736) or from one or more other sources through a suitable interface, such as network interface 2720. The software may cause processor(s) 2701 to carry out one or more processes or one or more steps of one or more processes described or illustrated herein. Carrying out such processes or steps may include defining data structures stored in memory 2703 and modifying the data structures as directed by the software.

The memory 2703 may include various components (e.g., machine readable media) including, but are not limited to, a random access memory component (e.g., RAM 2704) (e.g., static RAM (SRAM), dynamic RAM (DRAM), ferroelectric random access memory (FRAM), phase-change random access memory (PRAM), etc.), a read-only memory component (e.g., ROM 2705), and any combinations thereof. ROM 2705 may act to communicate data and instructions unidirectionally to processor(s) 2701, and RAM 2704 may act to communicate data and instructions bidirectionally with processor(s) 2701. ROM 2705 and RAM 2704 may include any suitable tangible computer-readable media described below. In one example, a basic input/output system 2706 (BIOS), including basic routines that help to transfer information between elements within computer system 2700, such as during start-up, may be stored in the memory 2703.

Fixed storage 2708 is connected bidirectionally to processor(s) 2701, optionally through storage control unit 2707. Fixed storage 2708 provides additional data storage capacity and may also include any suitable tangible computer-readable media described herein. Storage 2708 may be used to store operating system 2709, executable(s) 2710, data 2711, applications 2712 (application programs), and the like. Storage 2708 can also include an optical disk drive, a solid-state memory device (e.g., flash-based systems), or a combination of any of the above. Information in storage 2708 may, in appropriate cases, be incorporated as virtual memory in memory 2703.

In one example, storage device(s) 2735 may be removably interfaced with computer system 2700 (e.g., via an external port connector (not shown)) via a storage device interface 2725. Particularly, storage device(s) 2735 and an associated machine-readable medium may provide non-volatile and/or volatile storage of machine-readable instructions, data structures, program modules, and/or other data for the computer system 2700. In one example, software may reside, completely or partially, within a machine-readable medium on storage device(s) 2735. In another example, software may reside, completely or partially, within processor(s) 2701.

Bus 2740 connects a wide variety of subsystems. Herein, reference to a bus may encompass one or more digital signal lines serving a common function, where appropriate. Bus 2740 may be any of several types of bus structures including, but are not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 2700 may also include an input device 2733. In one example, a user of computer system 2700 may enter commands and/or other information into computer system 2700 via input device(s) 2733. Examples of an input device(s) 2733 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 2733 may be interfaced to bus 2740 via any of a variety of input interfaces 2723 (e.g., input interface 2723) including, but are not limited to, serial, parallel, game port, USB, FIREWIRE, THUNDERBOLT, or any combination of the above.

In particular embodiments, when computer system 2700 is connected to network 2730, computer system 2700 may communicate with other devices, specifically mobile devices and enterprise systems, distributed computing systems, cloud storage systems, cloud computing systems, and the like, connected to network 2730. Communications to and from computer system 2700 may be sent through network interface 2720. For example, network interface 2720 may receive incoming communications (such as requests or responses from other devices) in the form of one or more packets (such as Internet Protocol (IP) packets) from network 2730, and computer system 2700 may store the incoming communications in memory 2703 for processing. Computer system 2700 may similarly store outgoing communications (such as requests or responses to other devices) in the form of one or more packets in memory 2703 and communicated to network 2730 from network interface 2720. Processor(s) 2701 may access these communication packets stored in memory 2703 for processing.

Examples of the network interface 2720 include, but are not limited to, a network interface card, a modem, and any combination thereof. Examples of a network 2730 or network segment 2730 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 2730, may employ a wired and/or a wireless mode of communication. In general, any network topology may be used.

Information and data can be displayed through a display 2732. Examples of a display 2732 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 2732 can interface to the processor(s) 2701, memory 2703, and fixed storage 2708, as well as other devices, such as input device(s) 2733, via the bus 2740. The display 2732 is linked to the bus 2740 via a video interface 2722, and transport of data between the display 2732 and the bus 2740 can be controlled via the graphics control 2721. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 2732, computer system 2700 may include one or more other peripheral output devices 2734 including, but are not limited to, an audio speaker, a printer, a storage device, and any combinations thereof. Such peripheral output devices may be connected to the bus 2740 via an output interface 2724. Examples of an output interface 2724 include, but are not limited to, a serial port, a parallel connection, a USB port, a FIREWIRE port, a THUNDERBOLT port, and any combinations thereof.

In some embodiments, in addition or as an alternative, computer system 2700 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, in some embodiments, reference to a computer-readable medium may encompass a circuit (such as an IC) storing software for execution, a circuit embodying logic for execution, or both, where appropriate. The present disclosure encompasses any suitable combination of hardware, software, or both.

In some embodiments, those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

In some embodiments, the various illustrative logical blocks, modules, and circuits described in connection with the embodiments disclosed herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. In some embodiments, a general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

In some embodiments, the steps of a method or algorithm described in connection with the embodiments disclosed herein may be embodied directly in hardware, in a software module executed by one or more processor(s), or in a combination of the two. In some embodiments, a software module may reside in RAM memory, flash memory, ROM memory, EPROM memory, EEPROM memory, registers, hard disk, a removable disk, a CD-ROM, or any other form of storage medium known in the art. In some embodiments, an exemplary storage medium is coupled to the processor such the processor can read information from, and write information to, the storage medium. In the alternative, the storage medium may be integral to the processor. In some embodiments, the processor and the storage medium may reside in an ASIC. The ASIC may reside in a user terminal. In the alternative, the processor and the storage medium may reside as discrete components in a user terminal.

In some embodiments, in accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, internet appliances, mobile smartphones, tablet computers, personal digital assistants, video game consoles, and vehicles. In some embodiments, those of skill in the art will also recognize that select televisions, video players, and digital music players with optional computer network connectivity are suitable for use in the system described herein. Suitable tablet computers, in various embodiments, include, but are not limited to, those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. In some embodiments, those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some embodiments, those of skill in the art will recognize that suitable personal computer operating systems include, by way of non-limiting examples, Microsoft® Windows®, Apple® Mac OS X®, UNIX®, and UNIX-like operating systems, such as GNU/Linux®. In some embodiments, the operating system is provided by cloud computing. In some embodiments, those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. In some embodiments, those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. In some embodiments, those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems, including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. In some embodiments, a computer program includes a sequence of instructions, executable by one or more processor(s) of the computing device's CPU, written to perform a specified task. In some embodiments, computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

In some embodiments, the functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language, such as Hypertext Markup Language (HTML), Extensible Hypertext Markup Language (XHTML), or extensible Markup Language (XML). In some embodiments, a web application is written to some extent in a presentation definition language, such as Cascading Style Sheets (CSS). In some embodiments, a web application is written to some extent in a client-side scripting language, such as Asynchronous Javascript and XML (AJAX), Flash® Actionscript, Javascript, or Silverlight®. In some embodiments, a web application is written to some extent in a server-side coding language, such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language, such as Structured Query Language (SQL). In some embodiments, a web application integrates enterprise server products, such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

Referring to FIG. 23, in a particular embodiment, an application provision system comprises one or more databases 2800 accessed by a relational database management system (RDBMS) 2810. In some embodiments, suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 2820 (such as Java servers, .NET servers, PHP servers, and the like) and one or more web servers 2830 (such as Apache, IIS, GWS and the like). In some embodiments, the web server(s) optionally expose one or more web services via app application programming interfaces (APIs) 2840. Via a network, such as the Internet, the system provides browser-based and/or mobile native user interfaces.

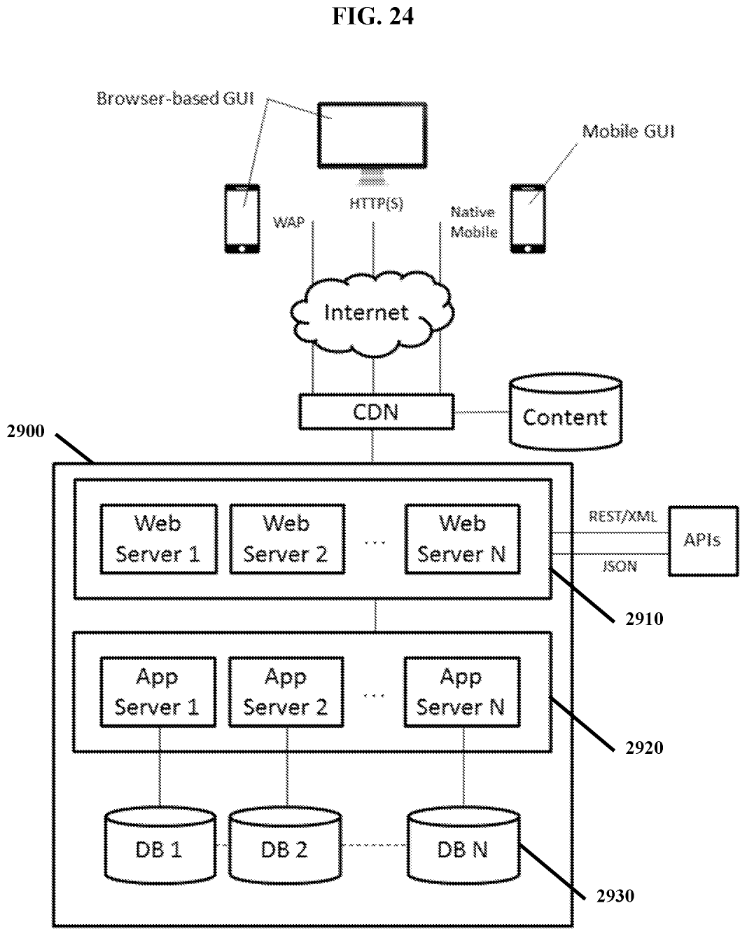

Referring to FIG. 24, in a particular embodiment, an application provision system alternatively has a distributed, cloud-based architecture 2900 and comprises elastically load balanced, auto-scaling web server resources 2910 and application server resources 2920 as well synchronously replicated databases 2930.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. In some embodiments, those of skill in the art will recognize that mobile applications are written in several languages. In some embodiments, suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

In some embodiments, suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

In some embodiments, those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® Dsi Shop.

### Standalone Application

In some embodiments, a computer program includes a standalone application, which is a program that is run as an independent computer process, not an add-on to an existing process, e.g., not a plug-in. In some embodiments, those of skill in the art will recognize that standalone applications are often compiled. In some embodiments, a compiler is a computer program(s) that transforms source code written in a programming language into binary object code, such as assembly language or machine code. In some embodiments, suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. In some embodiments, those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

In some embodiments, web browsers (also called Internet browsers) are software applications, designed for use with network-connected computing devices, for retrieving, presenting, and traversing information resources on the World Wide Web. In some embodiments, suitable web browsers include, by way of non-limiting examples, Microsoft®® Internet Explorer®, Mozilla® Firefox®, Google® Chrome, Apple® Safari®, Opera Software Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. In some embodiments, the software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform, such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of information. In various embodiments, suitable databases include, by way of non-limiting examples, relational databases, non-relational databases, object-oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Methods Utilizing a Computer

In some embodiments, the methods and software described herein can utilize one or more computers. In some embodiments, the computer can be used for managing customer and sample information, such as sample or customer tracking, database management, analyzing molecular profiling data, analyzing cytological data, storing data, billing, marketing, reporting results, storing results, or a combination thereof. In some embodiments, the computer can include a monitor or other graphical interface for displaying data, results, billing information, marketing information (e.g., demographics), customer information, or sample information. In some embodiments, the computer can also include means for data or information input. In some embodiments, the computer can include a processing unit and fixed or removable media or a combination thereof. In some embodiments, the computer can be accessed by a user in physical proximity to the computer, for example via a keyboard and/or mouse, or by a user that does not necessarily have access to the physical computer through a communication medium, such as a modem, an internet connection, a telephone connection, or a wired or wireless communication signal carrier wave. In some cases, the computer can be connected to a server or other communication device for relaying information from a user to the computer or from the computer to a user. In some cases, the user can store data or information obtained from the computer through a communication medium on media, such as removable media. In some embodiments, it is envisioned that data relating to the methods can be transmitted over such networks or connections for reception and/or review by a party. In some embodiments, the receiving party can be, but is not limited to, an individual, a health care provider or a health care manager. In one instance, a computer-readable medium includes a medium suitable for transmission of a result of an analysis of a biological sample. In some embodiments, the medium can include a result of a subject, wherein such a result is derived using the methods described herein.

In some embodiments, the entity obtaining the sample information can enter it into a database for the purpose of one or more of the following: inventory tracking, assay result tracking, order tracking, customer management, customer service, billing, and sales. Sample information can include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center, or any information suitable for storage in a database. In some embodiments, sample history can include, but is not limited to: age of the sample, type of sample, method of acquisition, method of storage, or method of transport.

In some embodiments, the database can be accessible by a customer, medical professional, insurance provider, or other third party. In some embodiments, database access can take the form of digital processing communication, such as a computer or telephone. In some embodiments, the database can be accessed through an intermediary, such as a customer service representative, business representative, consultant, independent testing center, or medical professional. In some embodiments, the availability or degree of database access or sample information, such as assay results, can change upon payment of a fee for products and services rendered or to be rendered. In some embodiments, the degree of database access or sample information can be restricted to comply with generally accepted or legal requirements for patient or customer confidentiality.

### Machine Learning

In some embodiments, the systems, methods, software, and platforms as described herein can comprise computer-implemented methods of supervised or unsupervised learning methods, including SVM, random forests, clustering algorithm (or software module), gradient boosting, logistic regression, and/or decision trees. In some embodiments, the machine learning methods as described herein can improve generation of suggestions based on recording and analyzing any of the identifiers, lab results, patient outcomes, or any other relevant medical information as described herein. In some cases, the machine learning methods can intentionally group or separate treatment options. In some embodiments, some treatment options can be intentionally clustered or removed from any one phase of the plurality of phases of the medical care encounter.

In some embodiments, supervised learning algorithms can be algorithms that rely on the use of a set of labeled, paired training data examples to infer the relationship between an input data and output data. In some embodiments, unsupervised learning algorithms can be algorithms used to draw inferences from training data sets to output data. In some embodiments, unsupervised learning algorithms can comprise cluster analysis, which can be used for exploratory data analysis to find hidden patterns or groupings in process data. One example of an unsupervised learning method can comprise principal component analysis. In some embodiments, principal component analysis can comprise reducing the dimensionality of one or more variables. In some embodiments, the dimensionality of a given variables can be at least 1, 5, 10, 50, 100, 200, 300, 400, 500, 600, 700, 800, 900, 1000, 1100, 1200 1300, 1400, 1500, 1600, 1700, 1800, or greater. In some embodiments, the dimensionality of a given variables can be at most 1800, 1600, 1500, 1400, 1300, 1200, 1100, 1000, 900, 800, 700, 600, 500, 400, 300, 200, 100, 50, 10 or less.

In some embodiments, the computer-implemented methods can comprise statistical techniques. In some embodiments, statistical techniques can comprise linear regression, classification, resampling methods, subset selection, shrinkage, dimension reduction, nonlinear models, tree-based methods, support vector machines, unsupervised learning, or any combination thereof.

In some embodiments, a linear regression can be a method to predict a target variable by fitting the best linear relationship between a dependent and independent variable. In some embodiments, the best fit can mean that the sum of all distances between a shape and actual observations at each point is the least. In some embodiments, linear regression can comprise simple linear regression and multiple linear regression. In some embodiments, a simple linear regression can use a single independent variable to predict a dependent variable. In some embodiments, a multiple linear regression can use more than one independent variable to predict a dependent variable by fitting a best linear relationship.

In some embodiments, a classification can be a data mining technique that assigns categories to a collection of data in order to achieve accurate predictions and analysis. In some embodiments, classification techniques can comprise logistic regression and discriminant analysis. Logistic regression can be used when a dependent variable is dichotomous (binary). In some embodiments, logistic regression can be used to discover and describe a relationship between one dependent binary variable and one or more nominal, ordinal, interval or ratio-level independent variables. In some embodiments, a resampling can be a method comprising drawing repeated samples from original data samples. In some embodiments, a resampling can involve a utilization of a generic distribution tables in order to compute approximate probability values. In some embodiments, a resampling can generate a unique sampling distribution on a basis of an actual data. In some embodiments, a resampling can use experimental methods, rather than analytical methods, to generate a unique sampling distribution. In some embodiments, resampling techniques can comprise bootstrapping and cross-validation. In some embodiments, bootstrapping can be performed by sampling with replacement from original data and take “not chosen” data points as test cases. In some embodiments, cross validation can be performed by split training data into a plurality of parts.

In some embodiments, a subset selection can identify a subset of predictors related to a response. In some embodiments, a subset selection can comprise best-subset selection, forward stepwise selection, backward stepwise selection, hybrid method, or any combination thereof. In some instances, shrinkage fits a model involving all predictors, but estimated coefficients are shrunken towards zero relative to the least squares estimates. In some embodiments, this shrinkage can reduce variance. In some embodiments, a shrinkage can comprise ridge regression and a lasso. In some embodiments, a dimension reduction can reduce a problem of estimating n+1 coefficients to a simpler problem of m+1 coefficients, where m<n. It can be attained by computing n different linear combinations, or projections, of variables. Then these n projections are used as predictors to fit a linear regression model by least squares. In some embodiments, dimension reduction can comprise principal component regression and partial least squares. In some embodiments, a principal component regression can be used to derive a low dimensional set of features from a large set of variables. In some embodiments, a principal component used in a principal component regression can capture the most variance in data using linear combinations of data in subsequently orthogonal directions. In some embodiments, the partial least squares can be a supervised alternative to principal component regression because partial least squares can make use of a response variable in order to identify new features.

In some embodiments, a nonlinear regression can be a form of regression analysis in which observational data are modeled by a function which is a nonlinear combination of model parameters and depends on one or more independent variables. In some embodiments, a nonlinear regression can comprise a step function, piecewise function, spline, generalized additive model, or any combination thereof.

In some embodiments, tree-based methods can be used for both regression and classification problems. In some embodiments, regression and classification problems can involve stratifying or segmenting the predictor space into a number of simple regions. In some embodiments, tree-based methods can comprise bagging, boosting, random forest, or any combination thereof. In some embodiments, bagging can decrease a variance of prediction by generating additional data for training from the original dataset using combinations with repetitions to produce multistep of the same carnality/size as original data. In some embodiments, boosting can calculate an output using several different models and then average a result using a weighted average approach. In some embodiments, a random forest algorithm can draw random bootstrap samples of a training set. In some embodiments, support vector machines can be classification techniques. In some embodiments, support vector machines can comprise finding a hyperplane that best separates two classes of points with the maximum margin. In some embodiments, support vector machines can constrain an optimization problem such that a margin is maximized subject to a constraint that it perfectly classifies data.

In some embodiments, unsupervised methods can be methods to draw inferences from datasets comprising input data without labeled responses. In some embodiments, unsupervised methods can comprise clustering, principal component analysis, k-Mean clustering, hierarchical clustering, or any combination thereof.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

As used in the specification and claims, the singular forms “a”, “an,” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells, and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 15% of that number. The term “about” a range refers to that range minus 15% of its lowest value and plus 15% of its greatest value.

As used herein, the terms “treatment” or “treating” are used in reference to a pharmaceutical or other intervention regimen for obtaining beneficial or desired results in the recipient. Beneficial or desired results include, but are not limited to, a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Embodiments

Embodiment 1. A method for detecting the presence of a medical condition or disease for a subject, the method comprising: collecting a bodily fluid sample from a subject; optionally, preserving the sample at the time of collection by addition of a preservative; optionally, fractionating the sample; optionally, adding a preservative to the fractionated sample; selecting one more analytes in the sample including, but are not limited to, nucleic acid transcripts or genomic regions of interest; qualitatively or quantitatively detecting the selected analytes with an assay(s), wherein the assay(s) may include techniques involving, but are not limited to, biomolecule purification, biomolecule enrichment, biomolecule sequencing, PCR, quantitative PCR, isothermal amplification, mass spectrometry, antibody-based detection, or CRISPR and CRISPR-Cas systems, thereby generating data; using a computer to analyze the data to detect the presence or absence of the medical condition or disease or analyzing the data to generate a likelihood score for the medical condition or disease.

Embodiment 2. The method of Embodiment 1, wherein the bodily fluid is saliva.

Embodiment 3. The method of Embodiment 1 or 2, wherein multiple samples are collected from a subject in a longitudinal manner to create a personal health profile that can be monitored for changes indicative of changes in health.

Embodiment 4. The method of Embodiment 1 or 2, wherein signal changes caused by circadian cycles are distinguished from signal arising from a medical condition or disease state.

Embodiment 5. The method of Embodiment 1, wherein a preservative is added to the sample after collection.

Embodiment 6. The method of Embodiment 1, wherein a preservative is present in the collection vessel prior to collection.

Embodiment 7. The method of Embodiment 1, wherein the preservative includes at least one or more of the following: EDTA for the inhibition of nucleases; an Rnase inhibitor, such as but is not limited to, poly(vinylsulfonic acid, sodium salt), dUppAp, pdUppAp and pTppAp, ribonucleoside vanadyl complexes, aurintricarboxylic acid, Rnasin, SUPERaseIN or similar; agents to prevent microbial growth, such as, but are not limited to, isothiazolinones and formaldehyde releasers, such as Germall Plus, DMDM hydantoin, imadozolidinyl urea, diazolidinyl urea, and Proclin 300; nucleic acid denaturing agents; protein denaturing agents, such as but are not limited to, detergents, urea, guanidinium thiocyanate, guanidinium hydrochloride; agents for sequestration of catabolic proteins; a buffering agent; a detergent; a reducing agent; an antioxidant; a cryoprotectant; or salt osmolytes.

Embodiment 8. The method of Embodiment 1, wherein the sample is fractionated post-collection.

Embodiment 9. The method of Embodiment 1, wherein the sample is fractioned into two or more parts through the application of a centrifugal force, and each fraction is removed separately, and where the fractions isolated may include a cell free fraction and a cell containing fraction.

Embodiment 10. The method of Embodiment 1, wherein the sample is fractioned via filtration.

Embodiment 11. The method of Embodiment 10, wherein the mechanism for filtration is integrated into the device and for which: one or more filters may be used. Multiple filters may be arranged in order by pore size to allow successively smaller species to pass through. Filtration is achieved through the application of mechanical force, centrifugal force, or vacuum, or through capillary action. Optionally, a preservative, such as that described in Embodiment 7, is added to the filtrate at the time of or shortly after filtration. Optionally, a preservative, such as that described in Embodiment 7, is added to the retentate.

Embodiment 12. The method of Embodiment 1, wherein saliva is fractionated to generate cell-free and cell containing portions using the methods described in any one of Embodiment 9-11.

Embodiment 13. The method of Embodiment 1, wherein at least one class of analytes comprise RNA.

Embodiment 14. The method of Embodiment 1, wherein at least one class of analytes comprise cell-free RNA.

Embodiment 15. The method of Embodiment 13, wherein the RNA is selected from the group consisting of: mRNA, small RNA, miRNA, snoRNA, snRNA, rRNAs, tRNA, tRNA fragments, siRNA, hnRNA, long non-coding RNA, shRNA, fragments thereof, and any combination thereof.

Embodiment 16. The method of embodiment 14, wherein the RNA is selected from the group consisting of: mRNA, small RNA, miRNA, snoRNA, snRNA, IRNAs, tRNA, tRNA fragments, siRNA, hnRNA, long non-coding RNA, shRNA, fragments thereof, and any combination thereof.

Embodiment 17. The method of Embodiment 1, wherein at least one class of analytes comprise DNA.

Embodiment 18. The method of Embodiment 1, wherein at least one class of analytes comprise cell free DNA.

Embodiment 19. The method of Embodiment 1, wherein the analytes are endogenous (originating from the subject), exogenous (e.g., subject's microbiome), or a mixture thereof.

Embodiment 20. The method of Embodiment 1, wherein at least one class of analytes comprise proteins.

Embodiment 21. The method of Embodiment 1, wherein at least one class of analytes comprise small molecules.

Embodiment 22. The method of Embodiment 1, wherein at least one class of analytes comprise hormones.

Embodiment 23. The method of Embodiment 1, wherein at least one class of analytes comprise metabolites.

Embodiment 24. The method of Embodiment 1, wherein at least one class of analytes comprise cells (endogenous or exogenous).

Embodiment 25. The method of Embodiment 1, wherein the disease is cancer.

Embodiment 26. The method of Embodiment 1, wherein the disease is breast cancer.

Embodiment 27. The method of Embodiment 26, wherein the bodily fluid is saliva.

Embodiment 28. The method of Embodiment 27, wherein the sample is fractionated to obtain cell-free saliva.

Embodiment 29. The method of Embodiment 26, wherein at least one analyte class is RNA.

Embodiment 30. The method of Embodiment 26, wherein at least one analyte class is cell-free RNA.

Embodiment 31. The method of Embodiment 29 or 30, wherein the RNA is analyzed using sequencing.

Embodiment 32. The method of Embodiment 31, wherein the sequencing is multiplexed.

Embodiment 33. The method of Embodiment 31, wherein the sequencing is high throughput.

Embodiment 34. The method of Embodiment 31, wherein unique molecular identifiers (UMIs) are used to identify a single RNA species that is represented multiple times.

Embodiment 35. The method of Embodiment 29 or 30, wherein the RNA in analyzed using PCR.

Embodiment 36. The method of Embodiment 29 or 30, wherein the RNA in analyzed using microarrays.

Embodiment 37. The method of Embodiment 26, wherein the patient has dense breast tissue.

Embodiment 38. The method of Embodiment 13 or 14, wherein tissue-specific contributions to the RNA profile are determined.

Embodiment 39. The method of Embodiment 38, wherein tissue-specific contributions to the RNA profile are subtracted either through the assay or computationally to distinguish the signal from the disease or medical condition.

Embodiment 40. The method of Embodiment 38, wherein the tissue-specific contributions are directly used to identify the presence of disease or medical condition.

Embodiment 41. The method of Embodiment 25, wherein tissue-specific contributions to the RNA profile are used to identify the cancer tissue of origin.

Embodiment 42. The method of Embodiment 1, wherein the disease is an infectious disease.

Embodiment 43. The method of Embodiment 1, wherein the disease or medical condition pertains to the brain or neurological system.

Embodiment 44. The method of Embodiment 1, wherein the medical condition pertains to the brain or neurological system.

Embodiment 45. The method of Embodiment 1, wherein the medical condition is pregnancy.

Embodiment 46. The method of Embodiment 1, wherein the medical condition is organ trauma or injury.

Embodiment 47. The method of Embodiment 1, wherein the disease is an autoimmune disease.

Embodiment 48. The method of Embodiment 1, wherein the disease is metabolic in nature.

Embodiment 49. The method of Embodiment 1, wherein the disease is of the endocrine system.

Embodiment 50. The method of Embodiment 1, wherein the disease is of the gastrointestinal tract.

Embodiment 51. The method of Embodiment 1, wherein the subject is human.

Embodiment 52. The method of Embodiment 1, wherein the subject is non-human.

Embodiment 53. The method of Embodiment 1, wherein the sample is collected at home.

Embodiment 54. The method of Embodiment 1, wherein the sample is collected in a medical care facility.

Embodiment 55. The method of Embodiment 1, wherein the sample is collected at a dental practice or by a dental care practitioner.

Embodiment 56. The method of Embodiment 1, wherein the sample is collected by a veterinary practitioner.

Embodiment 57. The method of Embodiment 1, wherein the preservative contains some or all of the following: (1) a reducing agent, such as tris(2-carboxyethyl) phosphine hydrochloride, β-mercaptoethanol, or dithiothreitol, (2) an antioxidant, such as ascorbate or ascorbic acid, (3) an antibacterial agent, such as Proclin 300 or isothiazolinones, (4) buffering agents to maintain the pH between 4 and 9, (5) nuclease inhibitors, such as EDTA, aurintricarboxylic acid, RNaseIn, etc., (6) an osmolyte, such as betaine, and (7) a cryoprotectant.

Embodiment 58. The method of Embodiment 1, wherein the preservative contains denaturants, such as guanidine thiocyanate or urea, and one or more of the following: (1) EDTA, (2) buffering agents, (3) detergents, and (4) a reducing agent.

The method of Embodiment 1, where the analytes are the patient's DNA and cell-free salivary RNA and both genetic and transcriptomic analysis are used to detect the presence of a disease or medical condition.

The method of Embodiment 1, where more than one sample is collected from the patient and the samples are processed using different versions of the workflow described in Embodiment 1.

The method of Embodiment 1, wherein the sample is collected from a bodily site that is different from a bodily site of the disease or condition.

### EXAMPLES

The following examples are included for illustrative purposes only and are not intended to limit the scope of the invention.

### Example 1. Tissue Specific Transcripts and Tissue Enriched Transcripts in Saliva

GTExdata was analyzed to identify genes that are highly specific for a small group of tissues. Tissue specific transcripts were detected from multiple organs in patient saliva (FIG. 4). The greatest overlap observed was with blood and esophagus. Tissue specific transcripts in saliva demonstrated its potential for broad disease detection.

GTExdata was analyzed to identify genes that are enriched across multiple tissues. This analysis extends the ability to use saliva for analysis of a larger number of tissues (FIG. 5).

Transcript enrichment (e.g., identification of tissue enriched transcript) was at least partially calculated based on a correlation weighted entropy calculation:

$$CWE = - {\sum }_{i} ( ( {p}_{i} log {p}_{i} ) / max \{ {\sum }_{j} {r}_{ij} , 0 \} )$$

where:
i represents tissue types,
p represents the normalized TPM in a tissue,
rᵢⱼ is the Pearson correlation coefficient between tissue types i and j, sums are over all tissue types
   For the numerator entropy term, lower values are indicative of expression in fewer tissues. The denominator is a weighting factor for highly correlated tissues. This reduces the CWE for genes that are expressed in correlated groups of tissue types (e.g., brain sections). For tissue enriched transcripts, the lowest 30th percentile for entropy was chosen. If gene is present at 50% or more of the max value, it was included as tissue enriched.

### Example 2. Breast Cancer Proof of Concept Study

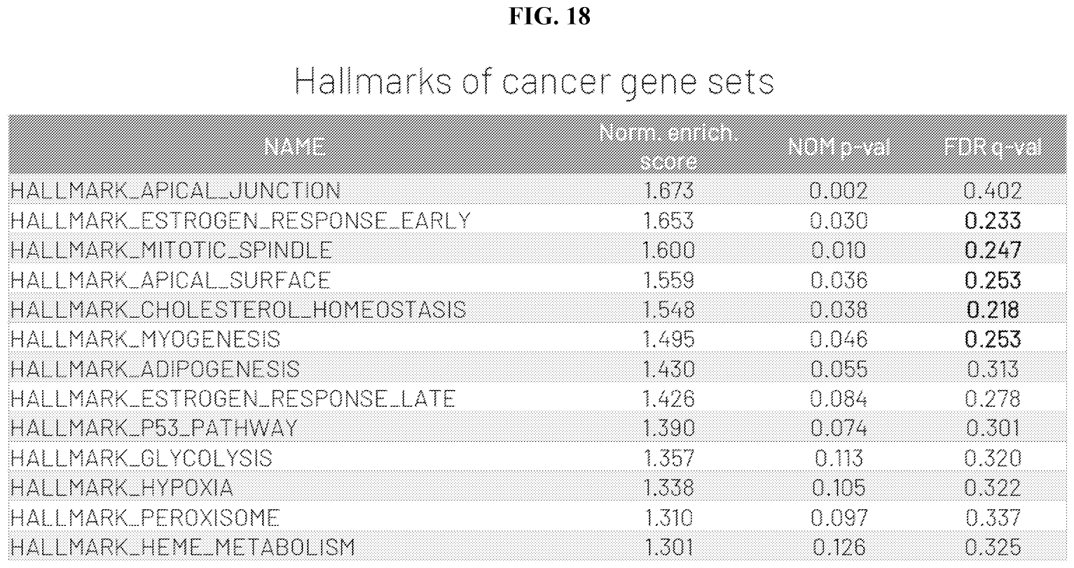

A clinical research study was conducted with over 2600 patients enrolled at two sites. A total of 2623 samples were collected. 115 breast cancer patients and 186 noncancer patients were analyzed in a 301 patient NGS study (FIG. 16). Genes were identified that are differentially abundant in the two groups. GSEA analysis further demonstrated that Hallmarks of Cancer gene lists are enriched in the cancer group (FIG. 18).

### Example 3. Detection of Breast Cancer

A method for detecting the presence of a medical condition or disease for a subject, the method comprising: obtaining saliva from a subject; purifying nucleic acids from the saliva; measuring levels of at least a portion of the nucleic acids using at least one or more of the following methods: sequencing, qPCR, microarrays; and using a computer and statistical methods to analyze the data to detect the presence or absence of the medical condition where all or subsets of the measurements may result in the detection of the condition. While this approach can be used to detect a multitude of diseases, this application is focused on methods and RNA signatures for breast cancer. The method may be used for patients in all risk groups who need to undergo screening or diagnostic workup for breast cancer. As such, it may be performed as a stand-alone test or paired with an imaging method, such as mammography, MRI, or ultrasound, to improve overall accuracy. Saliva collection may take place at home, in the field or at a medical facility. In all cases, the saliva must be collected, handled, and stored in a way that preserves the integrity of the analyte. In this case, the analytes of interest are nucleic acids that may be derived from whole or cell free saliva. The proper collection and preservation methods for preservation of nucleic acids is critical. It is desirable to use a method that inactivates nucleases to the greatest degree possible and preserves the nucleic acids over a broad range of temperature and for extended time periods. Other aspects of the sample collection, such as time or fasting state, may also play an important role and must be taken into consideration.

The preserved saliva sample may be fractioned to obtain cell-free saliva prior to analysis. The nucleic acids may be extracted from the sample using a variety of methods. Some methods are specific for RNA, some for DNA, and some methods allow isolation of both. In the case of RNA isolation, it is critical to use a method that removes any contaminating DNA and for DNA isolation, it is critical to use a method that removes contaminating RNA. Once isolated, the nucleic acids can be further analyzed using a variety of methods including, but are not limited to, sequencing, qPCR, and microarrays. In the case of genomic-scale technologies, it may be desirable to sub-select genes or regions for interest in either the assay or bioinformatically. This may be to reduce background noise or for reasons of cost. Regardless of the assay, any algorithm used to determine the presence of a disease or condition may use only a portion of the data to make the determination.

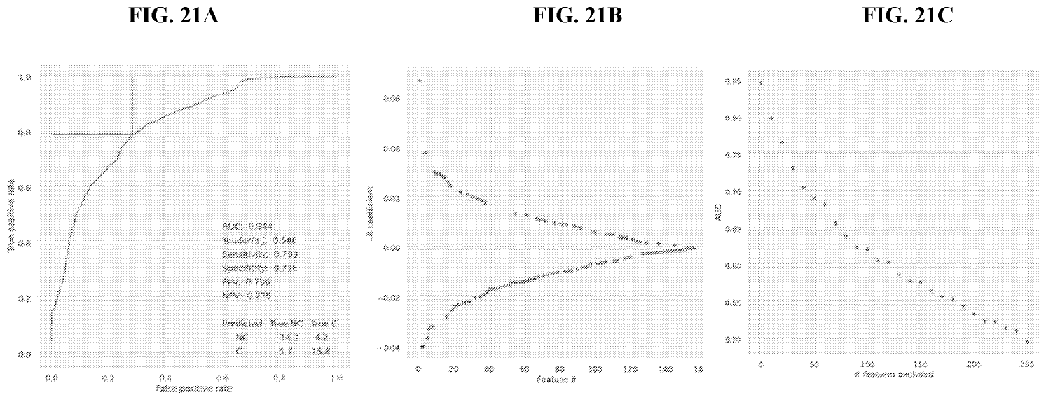

Example 3 illustrates breast cancer detection using RNA transcript found in cell free saliva. Using RNA-Seq data from a set of 20 breast cancer and 20 noncancer patients, machine learning logistic regression was used to identify genes that can be used in a classifier for breast cancer detection (Table 1). FIG. 21A shows ROC curves for 10-fold cross validated results, averaged over 100 repetitions with different train test splits. Cross-validation provided an estimate of how the model would perform on new data and guards against overfitting. Fitting all the data resulted in a model that used 157 genes, with logistic regression coefficients shown in FIG. 21B. While this set of 157 genes was used in the best performing classifier, it was also identified that an expanded set of 250 genes provided discrimination between cancer and noncancer patients. This set was identified by iteratively removing the most important features used by the classifier and re-fitting the classifier on the remaining genes. At each iteration, the performance of the classifier degraded. This process was repeated until the classification results were no better than random. It was found that this random performance occurred when 250 genes were removed (FIG. 21C). Based on these findings, it was concluded that expression levels of the genes listed in Table 1 can be predictive for the presence of breast cancer. It was noted that data from subsets of these genes may suffice for the classifier. A final classifier may use only a portion of these genes for prediction. Alternatively, the classifier may include many or all the genes with only a few being informative for disease prediction in any one sample.

TABLE 1
Exemplary gene list for detecting breast cancer

| | | | | |
|---|---|---|---|---|
| FAM110A | FOSB | NUP98 | DAB2IP | CBS |
| EIF3I | SERPINB1 | ITGA4 | MDH2 | STAG1 |
| BET1L | CGAS | CRACR2B | RPGRIP1 | CLPTM1 |
| TSPYL1 | TTLL12 | ZNF264 | CLSTN1 | PHTF1 |
| CDK2 | SPP1 | FCGR2B | CCNL1 | RANBP2 |
| GPR42 | HELB | FANCD2 | SH3BP1 | SSR4 |
| ODR4 | CHRNA10 | ELAPOR1 | AUP1 | ZNF622 |
| TFG | SEMA6B | VCL | ZFC3H1 | PGS1 |
| KRT8 | GSAP | EHMT2 | KIAA1522 | INAVA |
| FOXQ1 | ITPR1 | RELA | CHD2 | FBXO9 |
| NUDT16L1 | ATG3 | LAMP1 | MRPL41 | C9orf16 |
| RAB5A | PTOV1-AS2 | PYGB | HSPD1 | ARNTL |
| HEXIM1 | CTDP1 | NR4A1 | SYNJ2 | EGFR |
| ACBD3 | KLHL6 | AP1G1 | PRRG4 | PLIN4 |
| DDA1 | RNF122 | GLIS2 | GK | LPCAT3 |
| KRT7 | CCDC12 | RIPK1 | PLK3 | ATR |
| FOXO3B | HECA | N4BP2L2 | NPIPB13 | CSF2RA |
| ST3GAL4 | HPS5 | BTBD2 | RASA2 | LRSAM1 |
| TRIM11 | INSR | RP11-7F17.8 | ABCA1 | LMBR1L |
| SEC61A2 | SLC39A13 | FAM157B | DBNL | SCLT1 |
| FFAR3 | PNPLA8 | TLE2 | CLASP1 | MED11 |
| BICD2 | RAB11FIP1 | COX5A | AGO3 | SRSF11 |
| JUN | GABBR1 | ABCC5 | NFKBIZ | ZNF524 |
| STK3 | PPIG | RP4-622L5.7 | PPTC7 | KDM4C |
| PTPN2 | GZF1 | SH3YL1 | ACSF3 | NARF |
| DDX50 | KIAA0930 | HIF1A | CD300LF | SMARCD2 |
| GLUD1 | SCAPER | GRAMD1C | ANAPC11 | PGK1 |
| THAP5 | TACC1 | KHSRP | BIN3 | CCDC124 |
| ADRM1 | FBXO11 | RAB24 | CFLAR | CORO1B |
| RASA4B | ZBTB7A | RNF144B | ARID4B | FEM1A |
| CLTA | ZNF267 | ZNF703 | SCAND1 | BTBD1 |
| LGALS8 | NABP1 | CRACDL | RIPK3 | NOSIP |
| NAP1L4 | POU2F1 | THAP4 | FNDC3B | ARL5B |
| ARFGAP3 | IL10RA | FARP2 | SENP7 | DLG5 |
| PIN1 | PICALM | CISH | MYBPC3 | KLHL15 |
| IFT122 | SAMD1 | TNFRSF10D | SMARCC1 | NFRKB |
| CCDC85B | GRINA | CIITA | SF3A2 | CYB5R3 |
| DGAT2 | SECISBP2 | GTF2H1 | LMF2 | GPATCH2L |
| FOXC1 | RASSF4 | C4A | IL4R | NBPF26 |
| TADA2B | STYXL1 | CDADC1 | ZC3H3 | SLC9A3R2 |
| SEC22B | PRR5L | FRY | ZNF333 | CLEC4D |
| KIAA0586 | PITPNA | BAG1 | CSNK2A1 | PLEKHA7 |
| NOL10 | LYRM1 | BCAR1 | MRPS15 | ZNF574 |
| BATF | ARHGEF16 | DNASEIL3 | DPYD | APBB3 |
| ZNF274 | PDAP1 | ERP29 | DIS3 | NADSYN1 |
| DELE1 | SRP68 | NSUN7 | SPATA13 | RAPGEF1 |
| SSBP2 | CD4 | ACOT11 | NOLC1 | GPX1 |
| HEATR1 | H2BC21 | BTBD9 | ENG | CD48 |
| WTAP | ESRP2 | ECI1 | KRT18 | EDRF1 |
| EML4 | NBN | SORBS2 | CEACAM4 | TBC1D22A |

### Example 4. Nucleic Acid Preservation

Through the example below, two things are demonstrated: addition of a denaturant, such as a chaotropic agent, to a biological sample preserved RNA in that sample for at least four days; and sample filtration could be used to prepare cell-free or cell-depleted RNA.

Saliva collected from multiple individuals was pooled and split into two fractions, A and B (see FIG. 8). Fraction A was spun, and the cell free saliva was harvested and split into two parts, A1 and A2. To A1 and A2, an equivalent volume of PBS (preservative (−) condition) and chaotropic agent (preservative (+) condition) were added, respectively. Fraction B was filtered using a syringe-based filter to remove cells. The filtrate was split into two parts, B1 and B2. To B1 and B2, an equivalent volume of PBS (− preservative condition) and a chaotropic agent (+ preservative condition) were added, respectively. Aliquots were removed from A1, A2, B1, and B2 on days 0, 1, 2, and 4 and frozen at −80° C. Samples were extracted and analyzed using qPCR of three genes, ACTB, FOSL2, and NAMPT.

The qPCR results from the experiments demonstrated that the conditions containing the denaturing chaotropic agent (preservative +) showed no decline in transcript level for any of the three genes, while the conditions containing PBS (preservative −) showed a decrease in transcript level over time (FIG. 9). Filtration resulted in similar levels of transcripts as cell free saliva preparation using centrifugation for ACT and higher levels for FOSL2 and NAMPT. Whole saliva was also included as a control and generally shows higher levels of all three transcripts.

### Example 5. Nucleic Acid Preservation with Filtration and Preservative Condition

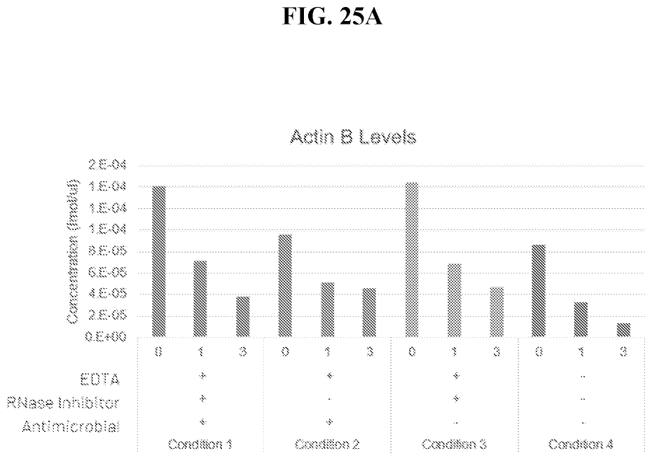

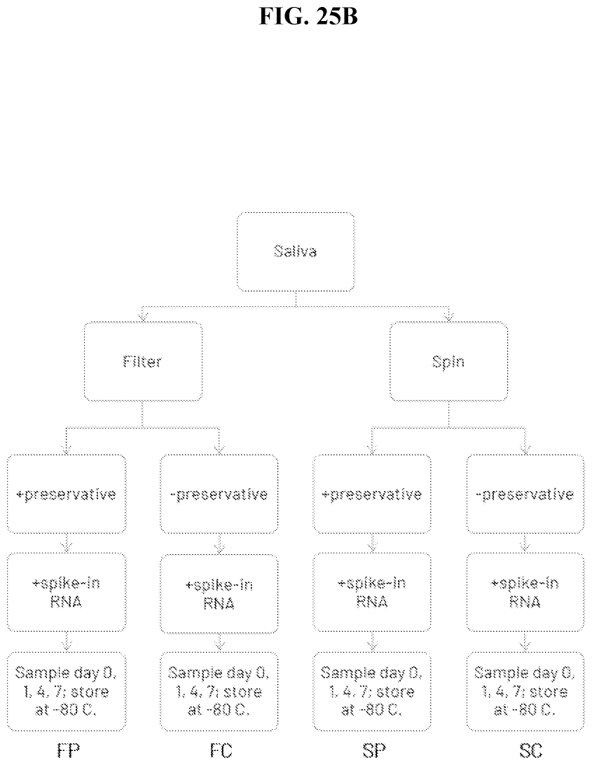

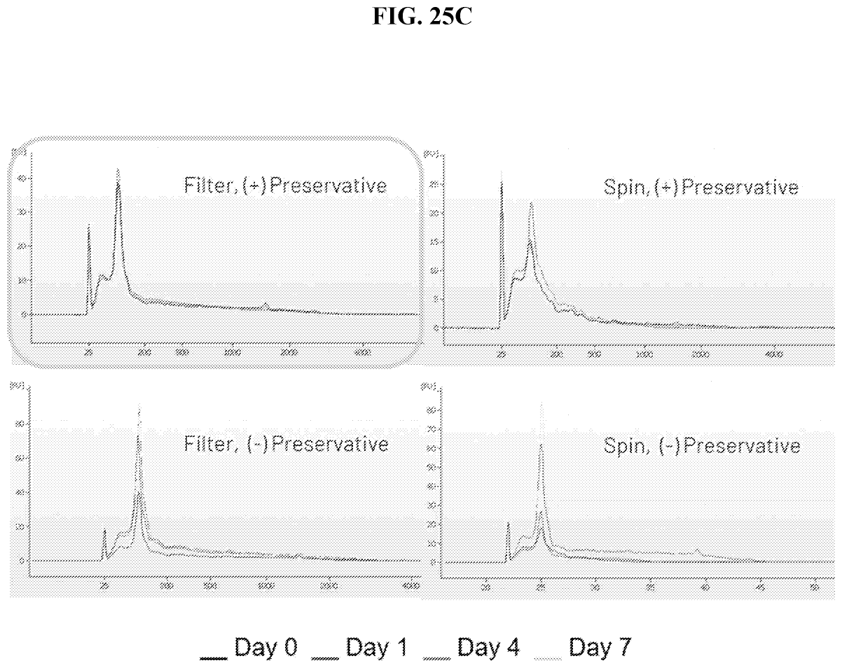

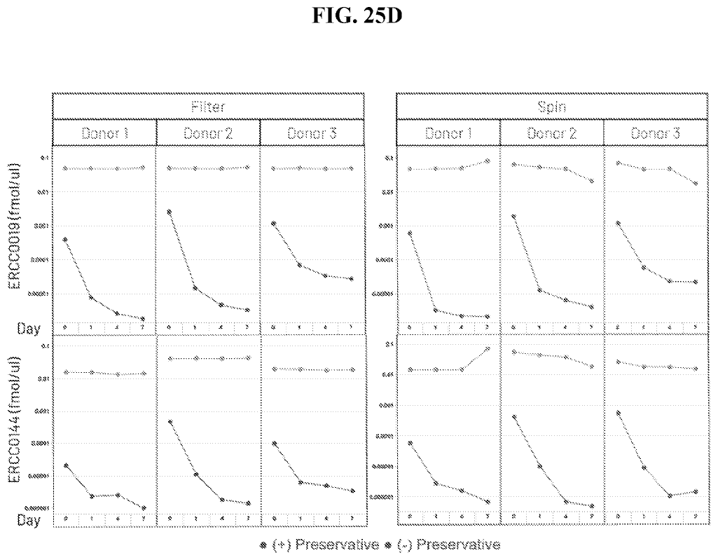

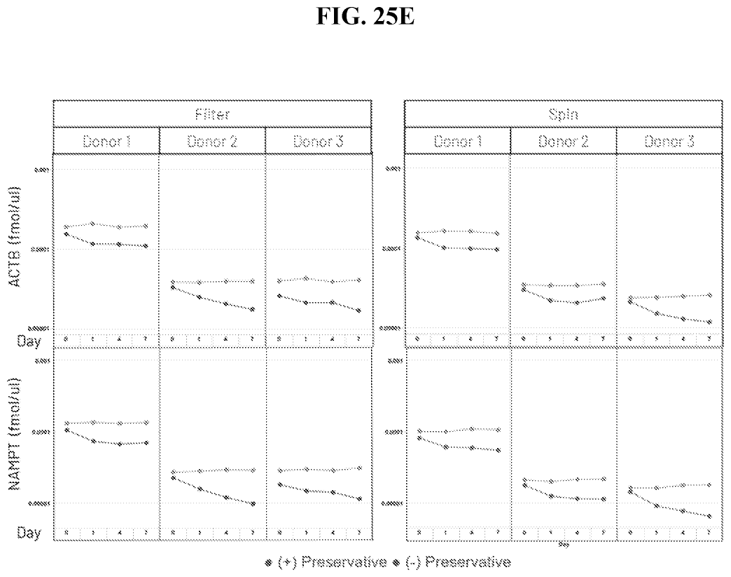

This example illustrates preserving whole saliva, where the whole saliva could be used for downstream analysis of nucleic acids, donor cells, microbiome, etc. The whole saliva was preserved with a combination of additives to stabilize components: nuclease inhibitors; anti-microbials to inhibit microbe growth; and fixatives to prevent cell lysis. FIG. 25A shows degradation of RNA encoding Actin B over three days. FIG. 25B shows a schematic diagram outlining a seven-day nucleic acid stability study of a method described herein for preserving nucleic acid. Saliva samples from three donors were obtained. The quality of the preserved nucleic acids was examined via qPCR for measuring abundance of synthetic spike-ins and endogenous genes. FIG. 25C shows an exemplary profile from one donor over seven days, illustrating that the filter and preservative combinatory condition showed the most stable profile over seven days. Conditions lacking preservative showed increase in signal over time (e.g., from bacterial growth). Filter and preservative condition were largely unchanged. FIG. 25D shows the preservation of spike-in control being preserved over seven days. Spike-in controls showed rapid degradation in the absence of preservative Spike-in controls had better stability in filtered samples than spun samples, even in the presence of preservative. Seven-day stability was observed. FIG. 25E shows stability of endogenous transcripts over seven days. Endogenous genes showed gradual degradation in the absence of preservative. Transcript levels were stable in the presence of preservative.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for detecting a disease or condition in a subject, the method comprising:
a) detecting the presence of at least one analyte or measuring the abundance of the at least one analyte in a sample from the subject; and
b) generating a score for the likelihood of the subject having the disease or condition or the subject developing the disease or condition,
wherein the sample is from a site of sample collection that is different from a site of the disease or condition, and
wherein the presence of the at least one analyte or the abundance of the at least one analyte in the sample correlates with the presence of the at least one analyte, the abundance of the at least one analyte in the site of the disease or condition, or a consequence of the disease or condition.

2. The method of claim 1, wherein prior to a), the method comprises preserving the sample.

3. The method of claim 2, wherein the preserving the sample comprises contacting the sample with a preservative comprising at least one of the following: ethylenediaminetetraacetic acid (EDTA); an RNase inhibitor; an anti-microbial; a denaturing agent; an agent that inhibits nuclease activity; a sequestration agent; a buffering agent; a salt; an osmolyte; or a combination thereof.

4. The method of claim 3, wherein the denaturing agent comprises a nucleic acid denaturing agent or a protein denaturing agent.

5. The method of any one of claims 1 to 4, wherein prior to a), the method further comprises fractionating the sample.

6. The method of claim 5, wherein the fractionating comprises separating the sample into two or more subsets of sample.

7. The method of claim 6, wherein at least one of the two or more subsets of sample comprises a cell-containing fraction, wherein the cell-containing fraction comprises a cell originating from the subject or a cell not originating from the subject.

8. The method of claim 7, wherein the cell originating from the subject is a human cell.

9. The method of claim 7 or 8, wherein the cell not originating from the subject is a non-human cell.

10. The method of claim 9, wherein the non-human cell comprises microbial cells.

11. The method of claim 9 or 10, wherein the non-human cell comprises bacterial cells.

12. The method of any one of claims 9 to 11, wherein the non-human cell comprises fungal cells.

13. The method of any one of claims 9 to 12, wherein the non-human cell comprises archaeal cells.

14. The method of any one of claims 6 to 13, wherein at least one of the two or more subsets of sample comprises a cell-free fraction.

15. The method of any one of claims 6 to 14, wherein the fractionating comprises centrifuging the sample or filtrating the sample.

16. The method of any one of claims 1 to 15, wherein the sample comprises a biofluid.

17. The method of claim 16, wherein the biofluid comprises blood, serum, plasma, saliva, urine, sweat, tears, breast milk, colostrum, semen, or cerebrospinal fluid.

18. The method of claim 17, wherein the biofluid comprises saliva.

19. The method of any one of claims 1 to 18, wherein the at least one analyte comprises a cell-free analyte.

20. The method of any one of claims 1 to 19, wherein the at least one analyte comprises a nucleic acid.

21. The method of claim 20, wherein the nucleic acid comprises a cell-free RNA.

22. The method of claim 20 or 21, wherein the nucleic acid comprises mRNA, small RNA, miRNA, snoRNA, snRNA, rRNAs, tRNA, siRNA, hnRNA, long non-coding RNA, shRNA, fragments thereof, or a combination thereof.

23. The method of any one of claims 1 to 22, wherein the at least one analyte comprises a polypeptide.

24. The method of claim 23, wherein the polypeptide is a protein.

25. The method of claim 23 or 24, wherein the polypeptide is a metabolite.

26. The method of any one of claims 1 to 25, wherein the at least one analyte comprises a small molecule.

27. The method of any one of claims 1 to 26, wherein the at least one analyte comprises a metabolite.

28. The method of any one of claims 1 to 18, wherein the at least one analyte comprises a cell.

29. The method of any one of claims 1 to 28, wherein a) comprises sequencing the at least one analyte, wherein the at least one analyte comprises at least one nucleic acid.

30. The method of claim 29, wherein a) comprises hybridizing the at least one nucleic acid with a probe.

31. The method of any one of claims 1 to 30, wherein the disease or condition is cancer.

32. The method of claim 31, wherein the cancer is breast cancer.

33. The method of any one of claims 1 to 30, wherein the disease or condition is a neurological disease.

34. The method of any one of claims 1 to 30, wherein the disease or condition is an autoimmune disease.

35. The method of any one of claims 1 to 30, wherein the disease or condition is a metabolic disease.

36. The method of any one of claims 1 to 30, wherein the disease or condition is an endocrine disease.

37. The method of any one of claims 1 to 30, wherein the disease or condition is a digestive tract disease.

38. The method of any one of claims 1 to 30, wherein the disease or condition is an injury.

39. The method of any one of claims 1 to 30, wherein the disease or condition is pregnancy.

40. The method of any one of claims 1 to 39, wherein the score determines the origin of the disease or condition.

41. The method of any one of claims 1 to 40, wherein the at least one analyte is DNA or cell-free salivary RNA of the subject, and both genetic and transcriptomic analyses are used to detect the presence of the disease or condition in the subject.

42. The method of any one of claims 1 to 41, wherein multiple samples from the subject are processed using different versions of the workflow described in any one of claims 1 to 41.

43. The method of any one of claims 1 to 42, further comprising collecting the sample from the subject.

44. A method for detecting a disease or condition in a subject, the method comprising:
with a computer system comprising a hardware processor and a memory on which instructions are encoded to cause the hardware processor to perform the operations of:
detecting the presence of at least one analyte or measuring the abundance of the at least one analyte in a sample from a subject; and
generating a score for the likelihood of the subject having the disease or condition or the subject developing the disease or condition,
wherein the sample is from a site of sample collection that is different from a site of the disease or condition, and
wherein the presence of the at least one analyte or the abundance of the at least one analyte in the sample correlates with the presence of the at least one analyte, the abundance of the at least one analyte in the site of the disease or condition, or a consequence of the disease or condition.

45. The method of claim 44, further comprising a step of generating a machine learning model iteratively trained to detect the disease or condition in the sample.

46. The method of claim 44 or 45, further comprising a step of generating a machine learning model iteratively trained to generate the score for the likelihood of the subject having the disease or condition.

47. The method of any one of claims 44 to 46, further comprising a step of generating a machine learning model iteratively trained to generate the score for the likelihood of the subject developing the disease or condition.

48. The method of any one of claims 45 to 47, wherein the machine learning model comprises at least one of a XGBoost algorithm, a logistic regression model and a random forest algorithm.

49. An apparatus for detecting a disease or condition in a subject, the apparatus comprising:
a computer system comprising a hardware processor and a memory on which instructions are encoded to cause the hardware processor to perform the operations of:
detecting the presence of at least one analyte or measuring the abundance of the at least one analyte in a sample acquired from the subject; and
generating a score for the likelihood of the subject having the disease or condition or the subject developing the disease or condition,
wherein the sample is from a site of sample collection that is different from a site of the disease or condition, and
wherein the presence of the at least one analyte or the abundance of the at least one analyte in the sample correlates with the presence of the at least one analyte, the abundance of the at least one analyte in the site of the disease or condition, or a consequence of the disease or condition.

50. The apparatus of claim 49, wherein the hardware processor generates a machine learning model iteratively trained to detect the disease or condition in the sample.

51. The apparatus of claim 49 or 50, wherein the hardware processor generates a machine learning model iteratively trained to generate the score for the likelihood of the subject having the disease or condition.

52. The apparatus of any one of claims 49 to 51, wherein the hardware processor generates a machine learning model iteratively trained to generate the score for the likelihood of the subject developing the disease or condition.

53. The apparatus of any one of claims 50 to 52, wherein the machine learning model comprises at least one of a XGBoost algorithm, logistic regression model and a random forest algorithm.
